(19) United States
(12) Patent Application Publication
(10) Pub. No.: US 2007/0117818 A1
(43) Pub. Date: May 24, 2007

(54) Pyrimidinone compounds useful as kinase inhibitors

(76) Inventors: Masaichi Hasegawa, Ibaraki (JP); Mio Takada, Ibaraki (JP); Yoshiaki Washio, Ibaraki (JP)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/588,527

(22) PCT Filed: Feb. 3, 2005
(86) PCT No.: PCT/US05/02972

§ 371 Date: Aug. 4, 2006

Related U.S. Application Data
(60) Provisional application No. 60/542,090, filed on Feb. 4, 2004.

(51) Int. Cl. A61K 31/513; C07D 405/02; C07D 403/02

(52) U.S. Cl. 514/269; 544/320; 544/321

(57) ABSTRACT

This invention relates to newly identified compounds for inhibiting hYAK3 proteins and methods for treating diseases associated with the imbalance or inappropriate activity of hYAK3 proteins.

### FIELD OF THE INVENTION

This invention relates to newly identified compounds for inhibiting hYAK3 proteins and methods for treating diseases associated with the imbalance or inappropriate activity of hYAK3 proteins.

### BACKGROUND OF THE INVENTION

A number of polypeptide growth factors and hormones mediate their cellular effects through a signal transduction pathway. Transduction of signals from the cell surface receptors for these ligands to intracellular effectors frequently involves phosphorylation or dephosphorylation of specific protein substrates by regulatory protein serine/threonine kinases (PSTK) and phosphatases. Serine/threonine phosphorylation is a major mediator of signal transduction in multicellular organisms. Receptor-bound, membrane-bound and intracellular PSTKs regulate cell proliferation, cell differentiation and signalling processes in many cell types.

Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are potential targets for drug design.

A subset of PSTKs are involved in regulation of cell cycling. These are the cyclin-dependent kinases or CDKs (Peter and Herskowitz, Cell 1994: 79, 181-184). CDKs are activated by binding to regulatory proteins called cyclins and control passage of the cell through specific cell cycle checkpoints. For example, CDK2 complexed with cyclin E allows cells to progress through the G1 to S phase transition. The complexes of CDKs and cyclins are subject to inhibition by low molecular weight proteins such as p 16 (Serrano et al, Nature 1993: 366, 704), which binds to and inhibits CDK4. Deletions or mutations in p 16 have been implicated in a variety of tumors (Kamb et al, Science 1994: 264, 436-440). Therefore, the proliferative state of cells and diseases associated with this state are dependent on the activity of CDKs and their associated regulatory molecules. In diseases such as cancer where inhibition of proliferation is desired, compounds that inhibit CDKs may be useful therapeutic agents. Conversely, activators of CDKs may be useful where enhancement of proliferation is needed, such as in the treatment of immunodeficiency.

YAK1, a PSTK with sequence homology to CDKs, was originally identified in yeast as a mediator of cell cycle arrest caused by inactivation of the cAMP-dependent protein kinase PKA (Garrett et al, Mol Cell Biol. 1991: 11-6045-4052). YAK1 kinase activity is low in cycling yeast but increases dramatically when the cells are arrested prior to the S-G2 transition. Increased expression of YAK1 causes growth arrest in yeast cells deficient in PKA. Therefore, YAK1 can act as a cell cycle suppressor in yeast.

Our U.S. Pat. No. 6,323,318 describes two novel human homologs of yeast YAK1 termed hYAK3-2, one protein longer than the other by 20 amino acids. hYAK3-2 proteins (otherwise reported as REDK-L and REDK-S in Blood, 1 May 2000, Vol 95, No. 9, pp 2838) are primarily localized in the nucleus. hYAK-2 proteins (hereinafter simply referred as hYAK3 or hYAK3 proteins) are present in hematopoietic tissues, such as bone marrow and fetal liver, but the RNA is expressed at significant levels only in erythroid or erthropoietin (EPO)-responsive cells. Two forms of REDK cDNAs appear to be alternative splice products. Antisense REDK oligonucleotides promote erythroid colony formation by human bone marrow cells, without affecting colony-forming unit (CFU)-GM, CFU-G, or CFU-GEMM numbers. Maximal numbers of CFU-E and burst-forming unit-erythroid were increased, and CFU-E displayed increased sensitivity to suboptimal EPO concentrations. The data indicate that REDK acts as a brake to retard erythropoiesis. Thus inhibitors of hYAK3 proteins are expected to stimulate proliferation of cells in which it is expressed. More particularly, inhibitors of hYAK3 proteins are useful to treat or prevent diseases of the erythroid and hematopoietic systems mediated the imbalance or inappropriate activity of hYAK3 proteins, including but not limited to, anemias due to renal insufficiency or to chronic disease, such as autoimmunity, HIV, or cancer, and drug-induced anemias, myelodysplastic syndrome, aplastic anemia and myelosuppression, and cytopenia.

### SUMMARY OF THE INVENTION

In the first aspect, the present invention relates to a compound of the formula I, or a salt, solvate, or a physiologically functional derivative thereof 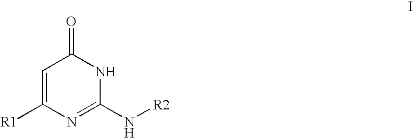
wherein R2 is a radical of the formula 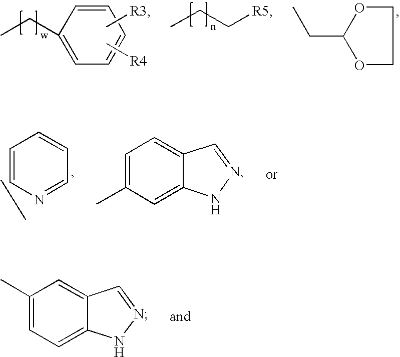
in which
n=1-2;
w=0-2;
R3 and R4 are independently hydrogen, hydroxy, —OR6, halogen, —SO₂NH₂, —OH, —C₁₋₆alkyl, —(C═O)—OEt, —NH(C═O)—CH₃, or a radical of the formula 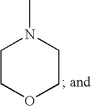
R5 is —NH₂, hydrogen, —OR6, —N(R6)(R7), or a radical of the formula 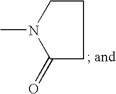
R1 is a radical of the formula 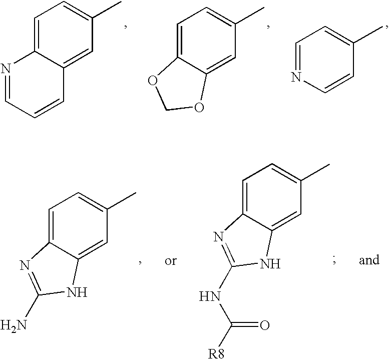
R8 is a radical of the formula 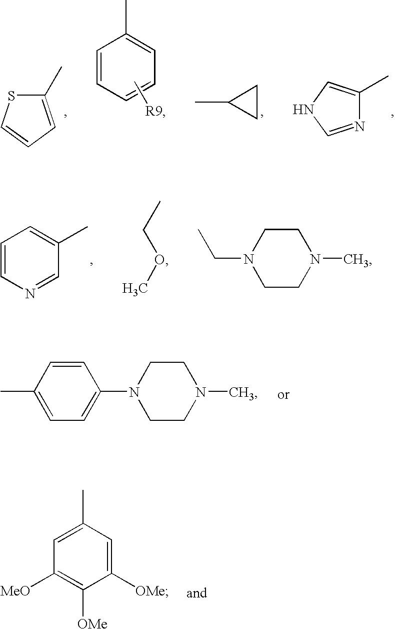
R9 is —NH(C═O)CH₃, —SO₂NH₂, —SO₂N(R6)(R7); and
R6 and R7 are independently C₁₋₆alkyl.

In a second aspect, the instant invention relates a method of inhibiting hYAK3 in a mammal; comprising, administering to the mammal a therapeutically effective amount of a compound of the formula I, or a salt, solvate, or a physiologically functional derivative thereof.

In a third aspect of the present invention, there is provided a pharmaceutical composition including a therapeutically effective amount of a compound of formula I, or a salt, solvate, or a physiologically functional derivative thereof and one or more of pharmaceutically acceptable carriers, diluents and excipients.

In a fourth aspect of the present invention, there is provided the use of a compound of formula I, or a salt, solvate, or a physiologically functional derivative thereof in the preparation of a medicament for use in the treatment or prevention of a disorder of the erythroid and hematopoietic systems mediated the imbalance or inappropriate activity of hYAK3 proteins, including but not limited to, anemias due to renal insufficiency or to chronic disease, such as autoimmunity, HIV, or cancer, and drug-induced anemias, myelodysplastic syndrome, aplastic anemia and myelosuppression, and cytopenia.

In a fifth aspect, the present invention relates to a method of treating or preventing diseases of the erythroid and hematopoietic systems, caused by the hYAK3 imbalance or inappropriate activity including, but not limited to, anemias due to renal insufficiency or to chronic disease, such as autoimmunity, HIV, or cancer, and drug-induced anemias, myelodysplastic syndrome, aplastic anemia and myelosuppression, and cytopenia; comprising, administering to a mammal a therapeutically effective amount of a compound of formula I, or a salt, solvate, or a physiologically functional derivative thereof and one or more of pharmaceutically acceptable carriers, diluents and excipients.

In a sixth aspect, the present invention relates to a method of treating or preventing anemias due to renal insufficiency or to chronic disease, such as autoimmunity, HIV, or cancer, and drug-induced anemias, myelodysplastic syndrome, aplastic anemia and myelosuppression, and cytopenia; comprising, administering to a mammal a therapeutically effective amount of a compound of formula I, or a salt, solvate, or a physiologically functional derivative thereof and one or more of pharmaceutically acceptable carriers, diluents and excipients.

### DETAILED DESCRIPTION

The present invention relates to a compound of the formula I, or a salt, solvate, or a physiologically functional derivative thereof, and its use in treating or preventing a disorder of the erythroid and hematopoietic systems mediated the imbalance or inappropriate activity of hYAK3 proteins 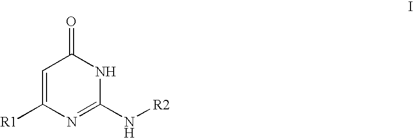
in which
R2 is a radical of the formula 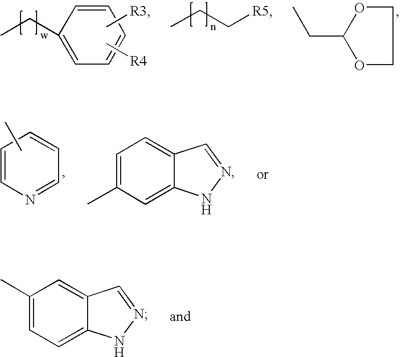
in which
n=1-2;
w=0-2;
R3 and R4 are independently hydrogen, hydroxy, —OR6, halogen, —SO₂NH₂, —OH, —C₁₋₆alkyl, —(C═O)—OEt, —NH(C═O)—CH₃, or a radical of the formula 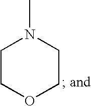
R5 is —NH₂, hydrogen, —OR6, —N(R6)(R7), or a radical of the formula 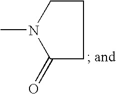
R1 is a radical of the formula 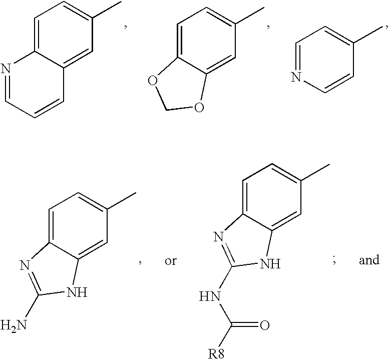
R8 is a radical of the formula 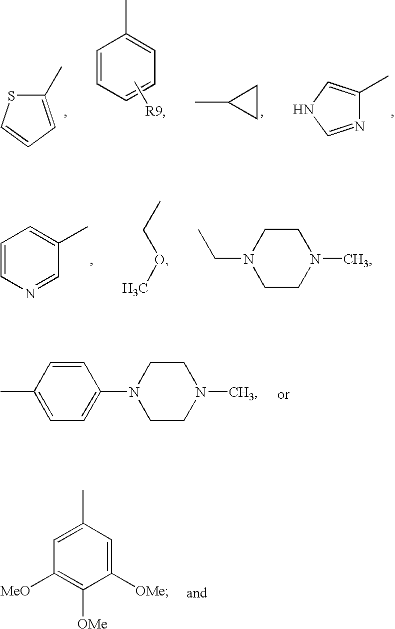
R9 is —NH(C═O)CH₃, —SO₂NH₂, —SO₂N(R6)(R7); and
R6 and R7 are independently C₁₋₆alkyl.

In a more preferred embodiment, R1 in a compound of formula I is a radical of the formula

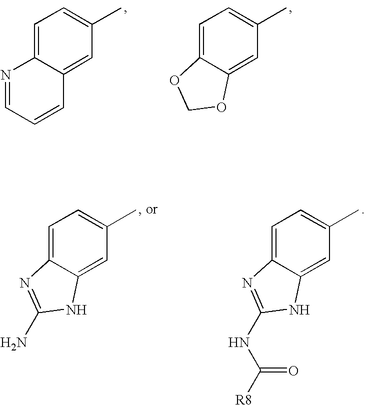

Yet in a further preferred embodiment, R1 in formula I is a radical of the formula

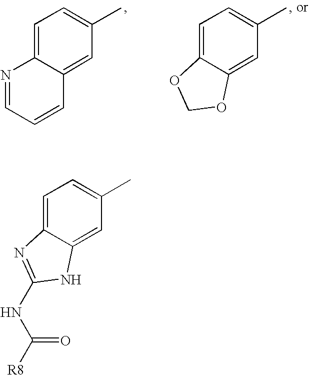

Yet in another further preferred embodiment, R1 is a radical of the formula

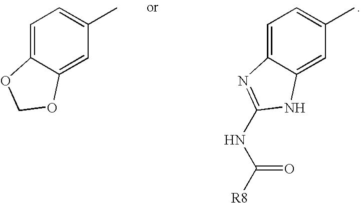

Yet in another further more preferred embodiment, R1 in formula I is a radical of the formula 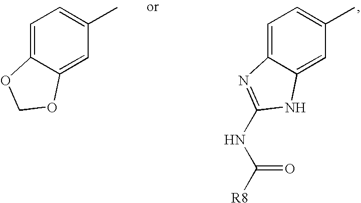
and R8 is 2-thienyl.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “alkyl” refers to a straight or branched chain hydrocarbon. Furthermore, as used herein, the term “C₁₋₆ alkyl” refers to an alkyl group as defined above containing at least 1, and at most 6, carbon atoms. Examples of branched or straight chained “C₁₋₆ alkyl” groups useful in the present invention include methyl, ethyl, n-propyl, isopropyl, isobutyl, n-butyl, t-butyl, n-pentyl, n-hexyl, and the like.

As used herein, the term “halogen” refers to fluorine (F), chlorine (Cl), bromine (Br), or iodine (I).

As used herein, the term “C₃₋₆ cycloalkyl” refers to a non-aromatic cyclic hydrocarbon ring having from three to six carbon atoms. Exemplary “C₃₋₆ cycloalkyl” groups include cyclopropyl, cyclobutyl, cyclopentyl, and cyclohexyl.

As used herein, the term “optionally” means that the subsequently described event(s) may or may not occur, and includes both event(s), which occur, and events that do not occur.

The present invention contemplates all possible tautomeric forms.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compound of formula I or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by formula I above as well as any wholly or partially equilibrated mixtures thereof. The present invention also covers the individual isomers of the compounds represented by the formulas above as mixtures with isomers thereof in which one or more chiral centers are inverted. Also, as stated above, it is understood that all tautomers and mixtures of tautomers are included within the scope of the compounds of formula I.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in the compound of formula I. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a compound of formula I, as well as salts, solvates and physiological functional derivatives thereof, may be administered as the raw chemical, it is possible to present the active ingredient as a pharmaceutical composition. Accordingly, the invention further provides pharmaceutical compositions (otherwise referred to as pharmaceutical formulations), which include therapeutically effective amounts of compounds of the formula I and salts, solvates and physiological functional derivatives thereof, and one or more pharmaceutically acceptable carriers, diluents, or excipients. The compounds of the formula I and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of the formula I, or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound of the formula I, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula I, and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula I, and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of formula I for the treatment of or prevention of diseases of the erythroid and hematopoietic systems, caused by hYAK3 imbalance or inappropriate activity including, but not limited to, neutropenia; cytopenia; anemias, including anemias due to renal insufficiency or to a chronic disease, such as autoimmunity, HIV or cancer, and drug-induced anemias; and myelosuppression will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a salt or solvate, or physiologically functional derivative thereof, may be determined as a proportion of the effective amount of the compound of formula I per se. It is envisaged that similar dosages would be appropriate for treatment of the other conditions referred to above.

Method of Preparation

Compounds of general formula I may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula I. Those skilled in the art will recognize if a stereocenter exists in compounds of formula I. Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

More particularly, the compounds of the formula I can be made by the process of either Scheme A or B or obvious variants thereof which appear throughout in the Examples below. Any person skilled in the art can readily adapt the processes of A, B and variants thereof, such the stoichemistry of the reagents, temperature, solvents, etc. to optimize the yield of the products desired.

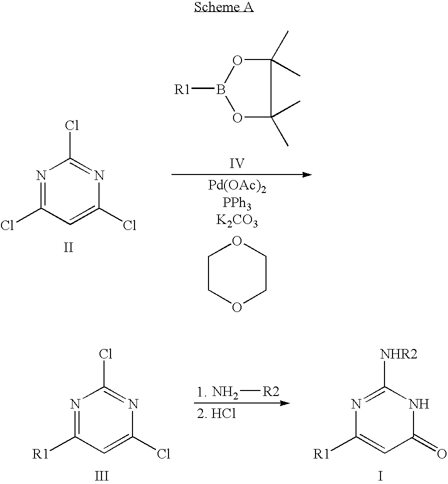

Briefly in Scheme A, 2,4,6-trichlorotriazine (II) is reacted with 4,4,5,5-tetramethyl[1,3,2]dioxaborolan of formula IV in the presence of Pd(OAc)₂, PPh₃, and K₂CO₃ in dioxane to afford a compound of formula III. One of two chloride groups in a compound of formula III is displaced by an amine R2NH₂, and further hydrolysis affords a compound of formula I.

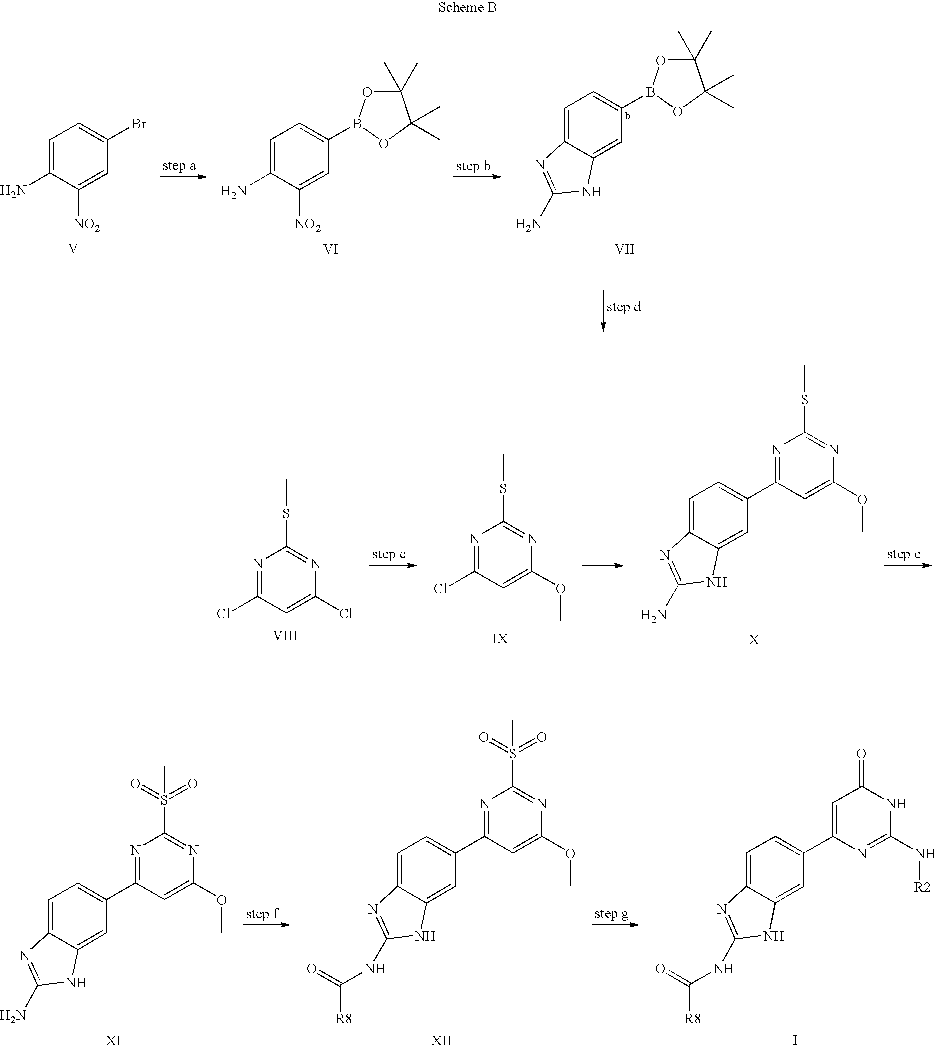

Briefly in Scheme B, in step (a), 2-nitro-4-(4,4,5,5-tetramethyl-1,3,2-dioxaborolan-2-yl)aniline (VI) is made from bis(pinacolato)diboron and a compound of formula V in the presence of palladium(II) acetate and potassium acetate. In step (b), compound VI is hydrogenated, and the product is reacted with cyanogen bromide to afford 5-(4,4,5,5-tetramethyl-1,3,2-dioxaborolan-2-yl)-1H-benzimidazol-2-amine (VII). Separately, in step (c), a chloride group in 4,6-dichloro-2-(methylthio)pyrimidine (VIII) is displaced with sodium methoxide to afford 4-chloro-6-(methyloxy)-2-(methylthio)pyrimidine (IX). In step (d), 4-chloro-6-(methyloxy)-2-(methylthio)pyrimidine is reacted with 5-(4,4,5,5-tetramethyl-1,3,2-dioxaborolan-2-yl)-1H-benzimidazol-2-amine in the presence of tetrakis(triphenylphosphine)palladium (0) and K₂CO₃ to afford 5-[6-(methyloxy)-2-(methylthio)-4-pyrimidinyl]-1H-benzimidazol-2-amine (X). The oxidation of 5-[6-(methyloxy)-2-(methylthio)-4-pyrimidinyl]-1H-benzimidazol-2-amine to 5-[6-(methyloxy)-2-(methylsulfonyl)-4-pyrimidinyl]-1H-benzimidazol-2-amine (XI) is carried out by hydrogen peroxide in the presence of sodium tungstate dihydrate in step (e). Acylation of nitrogen on compound of formula XI with R8(C═O)L, in which L is a leaving group such as hydroxy or halogen, is carried out via a conventional method (Step (f)), followed by displacement of sulfonylmethane group with an amine of formula H₂NR2 (Step (g)). (See below examples for examplification of Step (f) and Step (g)).

In Schemes A and B, R1, R2 and R8 are as previously defined.

### SPECIFIC EMBODIMENTS—EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
i. v. (intravenous); Hz (Hertz);
MHz (megahertz); mol (moles);
mmol (millimoles); rt (room temperature);
min (minutes); h (hours);
mp (melting point); TLC (thin layer chromatography);
Tr (retention time); RP (reverse phase);
MeOH (methanol); i-PrOH (isopropanol);
TEA (triethylamine); TFA (trifluoroacetic acid);
TFAA (trifluoroacetic anhydride); THF (tetrahydrofuran);
DMSO (dimethylsulfoxide); AcOEt (ethyl acetate);
DME (1,2-dimethoxyethane); DCM (dichloromethane);
DCE (dichloroethane); DMF (N,N-dimethylformamide);
DMPU (N,N′-dimethylpropyleneurea); CDI (1,1-carbonyldiimidazole);
IBCF (isobutyl chloroformate); HOAc (acetic acid);
HOSu (N-hydroxysuccinimide); HOBT (1-hydroxybenzotriazole);
mCPBA (meta-chloroperbenzoic acid; EDC (ethylcarbodimide hydrochloride);
BOC (tert-butyloxycarbonyl); FMOC (9-fluorenylmethoxycarbonyl);
DCC (dicyclohexylcarbodiimide); CBZ (benzyloxycarbonyl);
Ac (acetyl); atm (atmosphere);
TMSE (2-(trimethylsilyl)ethyl); TMS (trimethylsilyl);
TIPS (triisopropylsilyl); TBS (t-butyldimethylsilyl);
DMAP (4-dimethylaminopyridine); BSA (bovine serum albumin)
ATP (adenosine triphosphate); HRP (horseradish peroxidase);
DMEM (Dulbecco's modified Eagle medium);
HPLC (high pressure liquid chromatography);
BOP (bis(2-oxo-3-oxazolidinyl)phosphinic chloride);
TBAF (tetra-n-butylammonium fluoride);
HBTU (O-Benzotriazole-1-yl-N,N,N′,N′-tetramethyluronium hexafluorophosphate).
HEPES (4-(2-hydroxyethyl)-1-piperazine ethane sulfonic acid);
DPPA (diphenylphosphoryl azide);
fHNO3 (fumed HNO3); and
EDTA (ethylenediaminetetraacetic acid).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a Brucker AVANCE-400, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Low-resolution mass spectra (MS) were recorded on a JOEL JMS-AX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

### Example 1

### 2-(2-Amino-ethylamino)-6-quinolin-6-yl-3H-pyrimidin-4-one (Ia)

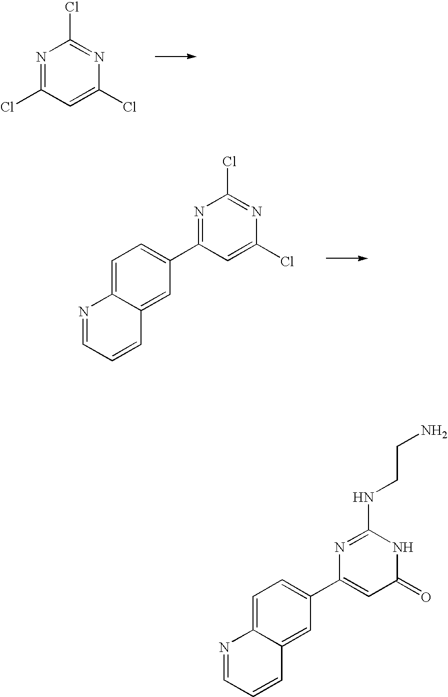

### a. 6-(2,6-Dichloro-pyrimidin-4-yl)-quinoline

To a mixture of 2,4,6-trichlorotriazine (1.25 g, 6.8 mmol), 6-(4,4,5,5-tetramethyl-[1,3,2]dioxaborolan-2-yl)-quinoline (1.74 g, 6.8 mmol), Pd(OAc)₂ (62 mg, 5 mol %) and PPh₃ (144 mg, 10 mol %), dioxane (30 mL) and 5M K₂CO₃ aq. (4 mL) were added and stirred at 100° C. for 2 hrs. The mixture was extracted with CH₂Cl₂ then the organic layer was washed with water. After drying over Na₂SO₄ and evaporation, the mixture was purified on SiO₂ column chromatography to give the title compound (1.06 g, 57%); MS (ESI) (M+H)⁺ 276.

### b. 2-(2-Amino-ethylamino)-6-quinolin-6-yl-3H-pyrimidin-4-one

To a solution of 6-(2,6-dichloro-pyrimidin-4-yl)-quinoline (110.4 mg, 0.4 mmol) in DMSO (8 mL), N-Boc ethylenediamine (189 μL, 1.2 mmol) was added and stirred at room temperature overnight. The mixture was added 2M HCl aq. (15 mL) and stirred at 85° C. overnight. The mixture was purified on SCX-SPE (BondElut® SCX (Varian Incorporated)) then on NH₂-SPE (BondElut® NH2 (Varian Incorporated) to give the title compound and its regio isomer (the yield not determined).

¹H-NMR (400 MHz, d₆-DMSO) δ 8.94(dd, 1H), 8.65(s, 1H), 8.47(d, 1H), 8.35(d, 1H), 8.05(d, 1H), 7.58(dd, 1H), 6.90(br, 1H), 6.32(s, 1H) and 2.78(t, 2H), 2H were overlapped with water, interchangeable 3H could not be detected; MS (ESI) (M+H)⁺ 282.

### Example 2

### 2-(2-Dimethylamino-ethylamino)-6-quinolin-6-yl-1H-pyrimidin-4-one (Ib)

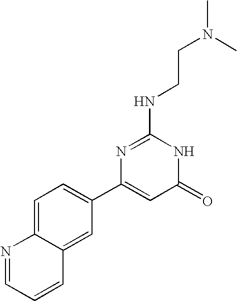

The title compound was prepared from 6-(2,6-dichloro-pyrimidin-4-yl)-quinoline and N,N-dimethylethylenediamine as described in example 1b.

¹H-NMR (400 MHz, d₆-DMSO) δ 10.92(br, 1H), 8.94(d, 1H), 8.67(s, 1H), 8.44(d, 1H), 8.36(dd, 1H), 8.05(d, 1H), 7.58(dd, 1H), 6.64(br, 1H), 6.34(s, 1H), 3.53(dt, 2H) and 2.23(s, 6H), 2H were overlapped with DMSO; MS (ESI) (M+H)⁺ 310.

### Example 3

### 2-(3-Methoxy-benzylamino)-6-quinolin-6-yl-1H-pyrimidin-4-one (Ic)

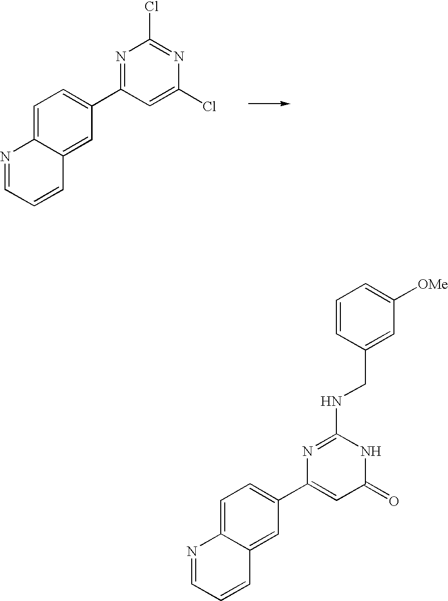

To a suspension of 6-(2,6-dichloro-pyrimidin-4-yl)-quinoline (34 mg, 0.12 mmol) in MeOH (1.3 mL), 4.1M NaOMe (0.03 mL) was added and stirred at 50° C. for 30 min. After evaporation, iPrOH (2 mL), 3-methoxybenzylamine (47 μL, 0.36 mmol) and 4M HCl-dioxane (0.05 mL) were added to the residue and heated by irradiation of microwave at 130° C. for 40 min. Additional 3-methoxybenzylamine (47 μL, 0.36 mmol) and 4M HCl-dioxane (0.3 mL) were added and heated at 130° C. for 80 min. The mixture was purified on SCX-SPE then on NH₂-SPE to give the title compound and its regio isomer (24 mg, 56%).

¹H-NMR (400 MHz, d₆-DMSO) δ 11.03(br, 1H), 8.93(dd, 1H), 8.65(d, 1H), 8.44(d, 1H), 8.34(d, 1H), 8.04(d, 1H), 7.57(dd, 1H), 7.28(dd, 1H), 7.17(br, 1H), 7.03-7.00(2H), 6.83(dd, 1H) 6.37(s, 1H), 4.63(d, 2H) and 3.25(s, 3H); MS (ESI) (M+H)⁺ 359.

### Example 4

### 2-{[2,6-Bis(methyloxy)phenyl]amino}-6-(6-quinolinyl)-4(1H)-pyrimidinone (Id)

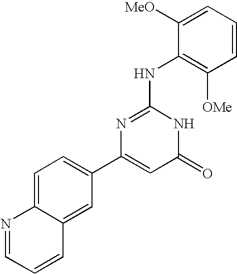

The title compound was prepared from 6-(2,6-dichloro-pyrimidin-4-yl)-quinoline and 2,6-dimethoxyaniline as described in example 3.

¹H-NMR (400 MHz, d₆-DMSO) δ 10.81 (br, 1H), 8.92(dd, 1H), 8.48(s, 1H), 8.36(d, 1H), 8.18(d, 1H), 8.03-7.94(2H), 7.56(dd, 1H), 7.28(t, 1H) 6.78(d, 2H), 6.39(s, 1H) and 3.79(s, 6H); MS (ESI) (M+H)⁺ 375.

### Example 5

### N-(5-{2-[(2-Chlorophenyl)amino]-6-oxo-1,6-dihydro-4-pyrimidinyl}-1H-benzimidazol-2-yl)-2-thiophenecarboxamide (Ie)

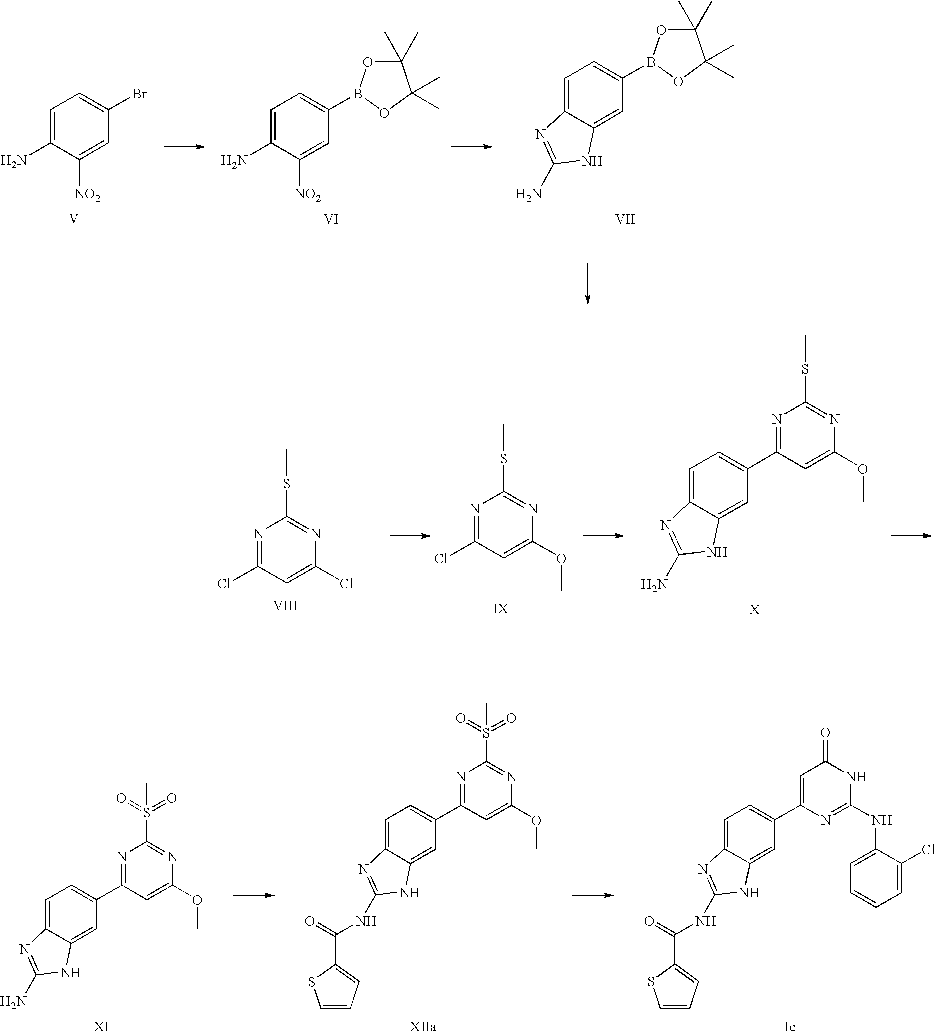

### a. 2-Nitro-4-(4,4,5,5-tetramethyl-1,3,2-dioxaborolan-2-yl)aniline (VI)

To a solution of 4-bromo-2-nitroaniline (10.0 g, 46.0 mmol), bis(pinacolato)diboron (12.8 g, 50.6 mmol) and potassium acetate (13.5 g, 138.0 mmol) in DMF (175 mL), palladium(II) acetate (309.8 mg, 1.38 mmol) was added and stirred at 85° C. for 5 hrs under argon. The mixture was concentrated, water was poured into it and extracted with CH₂Cl₂. The organic extract was dried over Na₂SO₄, and then concentrated. Recrystallizations from CH₂Cl₂/hexane gave the title compound (16.1 g, 92%). ¹H-NMR (400 MHz, d₆-DMSO) δ 8.27 (s, 1H), 7.70 (s, 2H), 7.55 (d, 1H), 6.98 (d, 1H) and 1.27 (s, 12H).

### b. 5-(4,4,5,5-Tetramethyl-1,3,2-dioxaborolan-2-yl)-1H-benzimidazol-2-amine (VII)

A flask was charged with 2-nitro-4-(4,4,5,5-tetramethyl-1,3,2-dioxaborolan-2-yl)aniline (17.3 g, 65.5 mmol) and palladium on carbon catalyst in MeOH (360 mL). Hydrogenation was carried out at 1 atm of hydrogen for 5 hrs. The mixture was filtered through celite, which was washed with MeOH. The solution was used for the next step without further purification. To the above solution, cyanogen bromide (8.4 g, 79.2 mmol) was added at room temperature. The mixture was stirred at room temperature for 1.5 hrs and then concentrated. Sat. NaHCO₃ was poured to the residue and the precipitated solid was collected by filtration, washed with EtOAc, and dried under vacuum to give the title compound (12.05 g, 71%).

¹H-NMR (400 MHz, d₆-DMSO) δ 8.27 (s, 1H), 7.70 (s, 2H), 7.55 (d, 1H), 6.97 (d, 1H) and 1.27 (s, 12H); MS (ESI) (M+H)⁺ 260.

### c. 4-Chloro-6-(methyloxy)-2-(methylthio)pyrimidine (IX)

To a solution of 4,6-dichloro-2-(methylthio)pyrimidine (7.8 g, 40 mmol) in MeOH (100 mL), NaOMe (4.1 M in MeOH, 10.2 mL) was added at 35° C. and stirred at same temperature for 1 hr. After evaporation, the mixture was extracted with CH₂Cl₂ and washed with water. The organic layer was dried over Na₂SO₄ then evaporated. The residue was purified on SiO₂ chromatography to give the title compound (5.8 g, 75%); MS (ESI) (M+H)⁺ 192.

### d. 5-[6-(Methyloxy)-2-(methylthio)-4-pyrimidinyl]-1H-benzimidazol-2-amine (X)

4-chloro-6-(methyloxy)-2-(methylthio)pyrimidine (3.75 g, 20.0 mmol), 5-(4,4,5,5-tetramethyl-1,3,2-dioxaborolan-2-yl)-1H-benzimidazol-2-amine (5.7 g, 22 mmol) and tetrakis(triphenylphosphine)palladium (0) (1.1 g, 1.0 mmol) were suspended in DMF (200 mL) and 2M K₂CO₃ aq (40 mL). The reaction vessel was sealed and heated in a microwave reactor to 180° C. for 5 minutes. After cooled to ambient temperature, the mixture was concentrated. Water was poured into the residue and the precipitated solid was collected by filtration, washed with EtOAc, and dried under vacuum to give the title compound (5.0 g, 87%). ¹H-NMR (400 MHz, d₆-DMSO) δ 10.9 (br, 1H), 7.94 (s, 1H), 7.76 (d, 1H), 7.16 (d, 1H), 7.03 (s, 1H), 6.44 (s, 2H), 3.94 (s, 3H) and 2.57 (s, 3H); MS (ESI) (M+H)⁺288.

### e. 5-[6-(Methyloxy)-2-(methylsulfonyl)-4-pyrimidinyl]-1H-benzimidazol-2-amine (XI)

To a solution of 5-[6-(methyloxy)-2-(methylthio)-4-pyrimidinyl]-1H-benzimidazol-2-amine (5.0 g, 17.5 mmol) and sodium tungstate dihydrate (285.1 mg, 0.86 mmol) in MeOH (122 mL), hydrogen peroxide (30% in water, 60 mL) was added at 0° C. The mixture was allowed to room temperature, stirred for 2 hrs. Then the mixture was cooled to 0° C. and sat. Na₂S₂O₃ aq. was added to it. A orange precipitate was collected, washed with EtOAc, and dried under vacuum to give the title compound (5.5 g, quant.). ¹H-NMR (400 MHz, d₆-DMSO) δ 10.9 (br, 1H), 8.02 (s, 1H), 7.85 (d, 1H), 7.60 (s, 1H), 7.20 (d, 1H), 6.52 (s, 2H), 4.05 (s, 3H) and 3.46 (s, 3H); MS (ESI) (M+H)⁺ 320.

### f. N-{5-[6-(methyloxy)-2-(methylsulfonyl)-4-pyrimidinyl]-1H-benzimidazol-2-yl}-2-thiophenecarboxamide (XIIa)

To a mixture of 5-[6-(methyloxy)-2-(methylsulfonyl)-4-pyrimidinyl]-1H-benzimidazol-2-amine (430 mg, 1.3 mmol), 2-thiophenecarboxylic acid (256.3 mg, 2.0 mmol) and triethylamine (538 μL) in DMF (13 mL) was added HBTU (758.5 mg, 2 mmol) and HOBt (149 mg, 1.1 mmol). The reaction mixture was stirred overnight at 35° C., then poured into water. A white precipitate was collected, washed with CH₂Cl₂, and dried under vacuum to give the title compound (345 mg, 62%). ¹H-NMR (400 MHz, d₆-DMSO) δ 12.4 (br, 2H), 8.38 (s, 1H), 8.10 (br, 1H), 8.07 (d, 1H), 7.90 (br, 1H), 7.72 (s, 1H), 7.56 (d, 1H), 7.24 (t, 1H), 4.08 (s, 3H) and 3.49 (s, 3H); MS (ESI) (M+H)⁺ 430.

### g. N-(5-{2-[(2-chlorophenyl)amino]-6-oxo-1,6-dihydro-4-pyrimidinyl}-1H-benzimidazol-2-yl)-2-thiophenecarboxamide (Ie)

N-{5-[6-(Methyloxy)-2-(methylsulfonyl)-4-pyrimidinyl]-1H-benzimidazol-2-yl}-2-thiophenecarboxamide (50 mg, 0.12 mmol) and o-cholroaniline (126 μL, 1.2 mmol) were dissolved in NMP (N-methylpyrroridinone, 0.4 mL) and HCl (4 N in dioxane, 0.12 mL). The reaction vessel was sealed and heated in a microwave reactor to 170° C. for 15 minutes. After cooled to ambient temperature, the mixture was neutralized with sat. NaHCO₃ aq. The mixture was purified on SCX-SPE then HPLC (11.4 mg, 21%). ¹H-NMR (400 MHz, d₆-DMSO)δ 12.42 (br, 2H), 8.46 (br, 2H), 8.08 (br, 2H), 7.85 (br, 1H), 7.81 (d, 1H), 7.55 (d, 1H), 7.47 (t, 2H), 7.21 (br, 1H), 7.18 (t, 1H) and 6.39 (s, 1H), interchangeable 1H could not be detected; MS (ESI) (M+H)⁺ 463.

### Example 6

### N-{5-[6-Oxo-2-(4-pyridinylamino)-1,6-dihydro-4-pyrimidinyl]-1H-benzimidazol-2-yl}-2-thiophenecarboxamide (If)

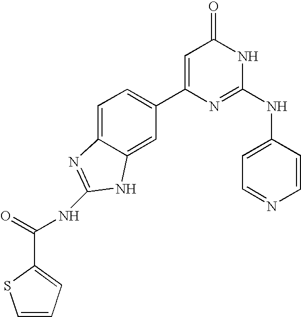

The title compound was prepared from N-{5-[6-(methyloxy)-2-(methylsulfonyl)-4-pyrimidinyl]-1H-benzimidazol-2-yl}-2-thiophenecarboxamide and 4-aminopyridine as in example 5g. ¹H-NMR (400 MHz, d₆-DMSO) δ 9.32 (d, 2H), 8.50 (s, 2H), 8.16 (s, 2H), 8.09 (s, 1H), 7.91 (br, 1H), 7.88 (d, 1H), 7.84 (s, 1H), 7.45 (d, 1H), 7.21 (t, 1H) and 6.94 (s, 1H), interchangeable 2H could not be detected; MS (ESI) (M+H)⁺ 430.

### Example 7

### N-[5-(2-{[3-(Aminosulfonyl)phenyl]amino}-6-oxo-1,6-dihydro-4-pyrimidinyl)-1H-benzimidazol-2-yl]-2-thiophenecarboxamide (Ig)

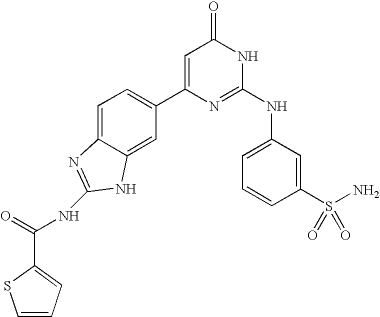

The title compound was prepared from N-{5-[6-(methyloxy)-2-(methylsulfonyl)-4-pyrimidinyl]-1H-benzimidazol-2-yl}-2-thiophenecarboxamide and 3-aminobenzenesulfonamide as in example 5g. ¹H-NMR (400 MHz, d₆-DMSO) δ 12.37 (br, 2H), 8.54 (br, 1H), 8.17 (s, 1H), 7.98 (m, 3H), 7.88 (br, 1H), 7.54 (t, 1H), 7.49 (d, 2H), 7.38 (s, 2H), 7.22 (br, 1H) and 6.46 (s, 1H), interchangeable 2H could not be detected; MS (ESI) (M+H)⁺ 508.

### Example 8

### N-{5-[6-Oxo-2-(phenylamino)-1,6-dihydro-4-pyrimidinyl]-1H-benzimidazol-2-yl}-2-thiophenecarboxamide (Ih)

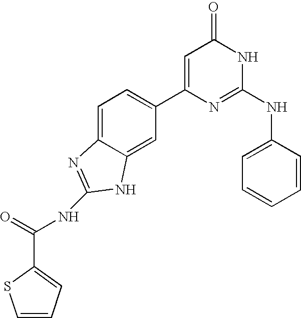

The title compound was prepared from N-{5-[6-(methyloxy)-2-(methylsulfonyl)-4-pyrimidinyl]-1H-benzimidazol-2-yl}-2-thiophenecarboxamide and aniline as in example 5g. ¹H-NMR (400 MHz, d₆-DMSO) δ 12.43 (br, 2H), 8.16 (s, 1H), 7.85 (d, 1H), 7.79 (d, 2H), 7.49 (d, 1H), 7.39 (t, 2H), 7.22 (s, 1H), 7.06 (t, 1H) and 6.38 (s, 1H), interchangeable 4H could not be detected; MS (ESI) (M+H)⁺ 429.

### Example 9

### N-(5-{2-[(3-Hydroxyphenyl)amino]-6-oxo-1,6-dihydro-4-pyrimidinyl}-1H-benzimidazol-2-yl)-2-thiophenecarboxamide (Ii)

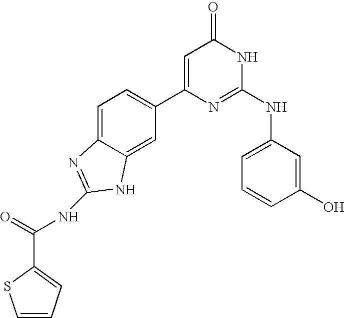

The title compound was prepared from N-{5-[6-(methyloxy)-2-(methylsulfonyl)-4-pyrimidinyl]-1H-benzimidazol-2-yl}-2-thiophenecarboxamide and 3-aminophenol as in example 5g. ¹H-NMR (400 MHz, d₆-DMSO) δ 12.43 (br, 2H), 9.40 (br, 1H), 8.15 (s, 1H), 8.05 (br, 1H), 7.88 (m, 2H), 7.49 (d, 1H), 7.28 (br, 2H), 7.21 (t, 1H), 7.15 (t, 1H), 6.45 (d, 1H) and 6.33 (s, 1H), interchangeable 2H could not be detected; MS (ESI) (M+H)⁺ 445.

### Example 10

### N-{5-[2-(1H-Indazol-6-ylamino)-6-oxo-1,6-dihydro-4-pyrimidinyl]-1H-benzimidazol-2-yl }-2-thiophenecarboxamide (Ij)

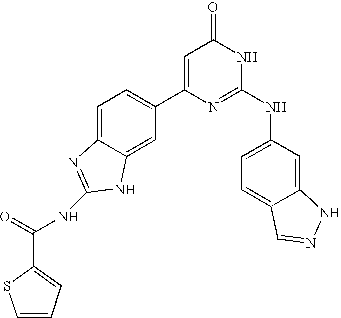

The title compound was prepared from N-{5-[6-(methyloxy)-2-(methylsulfonyl)-4-pyrimidinyl]-1H-benzimidazol-2-yl}-2-thiophenecarboxamide and 6-aminoindazole as in example 5g. ¹H-NMR (400 MHz, d₆-DMSO) δ 12.94 (s, 1H), 12.42 (br, 2H), 9.13 (br, 1H), 8.53 (br, 1H), 8.17 (s, 1H), 8.00 (s, 1H), 7.94 (d, 1H), 7.87 (br, 1H), 7.72 (d, 1H), 7.54 (d, 1H), 7.22 (br, 2H) and 6.41 (s, 1H), interchangeable 2H could not be detected; MS (ESI) (M+H)⁺ 469.

### Example 11

### N-[5-(2-{[4-(Methyloxy)phenyl]amino}-6-oxo-1,6-dihydro-4-pyrimidinyl)-1H-benzimidazol-2-yl]-2-thiophenecarboxamide (Ik)

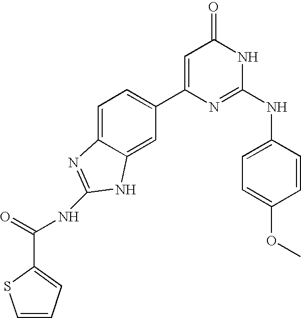

The title compound was prepared from N-{5-[6-(methyloxy)-2-(methylsulfonyl)-4-pyrimidinyl]-1H-benzimidazol-2-yl}-2-thiophenecarboxamide and p-anisidine as in example 5g. ¹H-NMR (400 MHz, d₆-DMSO) δ 12.47 (br, 1H), 8.72 (br, 1H), 8.32 (s, 1H), 7.82 (m, 2H), 7.66 (d, 2H), 7.47 (s, 1H), 7.22 (br, 1H), 6.97 (d, 2H), 6.19 (br, 1H) and 3.78 (s, 3H), interchangeable 3H could not be detected; MS (ESI) (M+H)⁺ 459.

### Example 12

### N-[5-(2-{[2-(Methyloxy)ethyl]amino}-6-oxo-1,6-dihydro-4-pyrimidinyl)-1H-benzimidazol-2-yl]-2-thiophenecarboxamide (II)

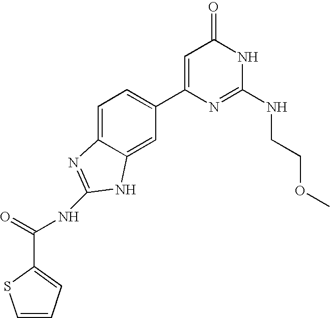

The title compound was prepared from N-{5-[6-(methyloxy)-2-(methylsulfonyl)-4-pyrimidinyl]-1H-benzimidazol-2-yl}-2-thiophenecarboxamide and 2-methoxyethylamine as in example 5g. ¹H-NMR (400 MHz, d₆-DMSO) δ 12.43 (br, 1H), 8.47 (s, 1H), 8.10 (s, 1H), 8.00 (br, 1H), 7.83 (m, 2H), 7.42 (d, 1H), 7.20 (t, 1H), 6.66 (br, 1H), 6.09 (s, 1H), 4.10 (s, 3H), 3.61 (q, 2H) and 3.58 (t, 2H), interchangeable 1H could not be detected; MS (ESI) (M+H)⁺ 411.

### Example 13

### N-[5-(2-{[2-(Methyloxy)phenyl]amino}-6-oxo-1,6-dihydro-4-pyrimidinyl)-1H-benzimidazol-2-yl]-2-thiophenecarboxamide (Im)

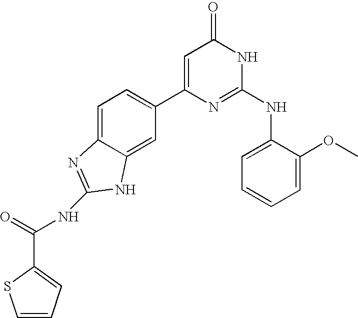

The title compound was prepared from N-{5-[6-(methyloxy)-2-(methylsulfonyl)-4-pyrimidinyl]-1H-benzimidazol-2-yl}-2-thiophenecarboxamide and o-anisidine as in example 5g. ¹H-NMR (400 MHz, d₆-DMSO) δ 12.43 (br, 2H), 11.24 (br, 1H), 8.62 (t, 1H), 8.49 (br, 1H), 8.15 (s, 1H), 8.07 (br, 1H), 7.86 (br, 1H), 7.85 (d, 1H), 7.48 (d, 1H), 7.23 (t, 1H), 7.10 (m, 3H), 6.34 (s, 1H) and 3.92 (s, 3H); MS (ESI) (M+H)⁺ 459.

### Example 14

### N-[5-(2-{[3-(Methyloxy)phenyl]amino}-6-oxo-1,6-dihydro-4-pyrimidinyl)-1H-benzimidazol-2-yl]-2-thiophenecarboxamide (In)

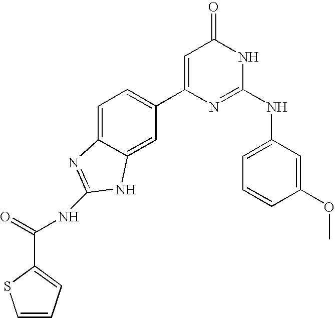

The title compound was prepared from N-{5-[6-(methyloxy)-2-(methylsulfonyl)-4-pyrimidinyl]-1H-benzimidazol-2-yl}-2-thiophenecarboxamide and m-anisidine as in example 5g. ¹H-NMR (400 MHz, d₆-DMSO) δ 12.43 (br, 2H), 8.13 (s, 1H), 7.86 (d, 2H), 7.61 (br, 1H), 7.49 (d, 1H), 7.25 (m, 3H), 6.63 (d, 1H), 6.37 (br, 1H) and 3.80 (s, 3H), interchangeable 3H could not be detected; MS (ESI) (M+H)⁺ 459.

### Example 15

### N-[5-(2-{[3-(Dimethylamino)propyl]amino}-6-oxo-1,6-dihydro-4-pyrimidinyl)-1H-benzimidazol-2-yl]-2-thiophenecarboxamide (Io)

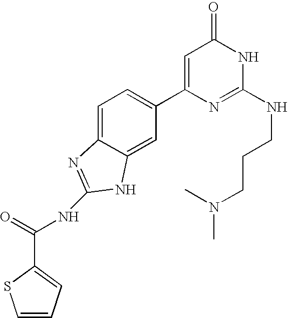

The title compound was prepared from N-{5-[6-(methyloxy)-2-(methylsulfonyl)-4-pyrimidinyl]-1H-benzimidazol-2-yl}-2-thiophenecarboxamide and 3-(dimethylamino)propylamine as in example 5g. ¹H-NMR (400 MHz, d₆-DMSO) δ 8.09 (s, 1H), 8.01 (br, 1H), 7.81 (m, 2H), 7.43 (d, 1H), 7.20 (t, 1H), 6.68 (br, 1H), 6.07 (s, 1H), 3.42 (br, 6H), 2.31 (t, 2H) and 1.71 (t, 2H), interchangeable 3H could not be detected, 2H were overlapped with water; MS (ESI) (M+H)⁺ 438.

### Example 16

### N-[5-(2-{[2-(2,4-Dichlorophenyl)ethyl]amino}-6-oxo-1,6-dihydro-4-pyrimidinyl)-1H-benzimidazol-2-yl]-2-thiophenecarboxamide (Ip)

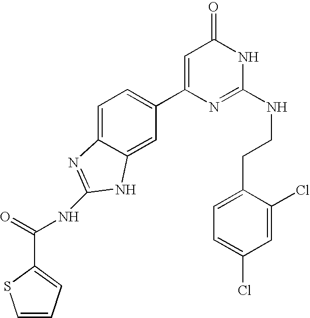

The title compound was prepared from N-{5-[6-(methyloxy)-2-(methylsulfonyl)-4-pyrimidinyl]-1H-benzimidazol-2-yl}-2-thiophenecarboxamide and 2,4-dichlorophenethylamine as in example 5g. ¹H-NMR (400 MHz, d₆-DMSO) δ 12.2 (br, 1H), 8.07 (s, 1H), 7.88 (br, 1H), 7.77 (d, 2H), 7.59 (s, 1H), 7.41 (m, 3H), 7.18 (s, 1H), 6.58 (br, 1H), 6.07 (s, 1H), 3.68 (q, 2H) and 3.05 (t, 2H), interchangeable 2H could not be detected; MS (ESI) (M+H)⁺ 525.

### Example 17

### N-[5-(2-{[2-(4-Morpholinyl)phenyl]amino}-6-oxo-1,6-dihydro-4-pyrimidinyl)-1H-benzimidazol-2-yl]-2-thiophenecarboxamide (Iq)

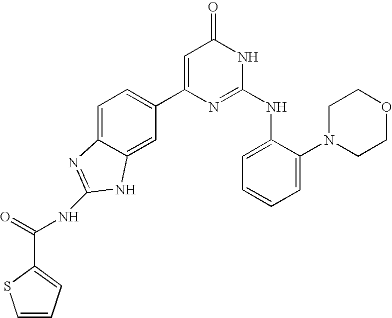

The title compound was prepared from N-{5-[6-(methyloxy)-2-(methylsulfonyl)-4-pyrimidinyl]-1H-benzimidazol-2-yl}-2-thiophenecarboxamide and 2-(4-morpholinyl)aniline as in example 5g. ¹H-NMR (400 MHz, d₆-DMSO) δ 12.44 (br, 2H), 11.99 (br, 1H), 8.58 (d, 1H), 8.45 (s, 1H), 8.15 (s, 1H), 7.83 (dd, 2H), 7.48 (d, 1H), 7.28 (t, 1H), 7.22 (br, 2H), 7.10 (dt, 2H), 6.35 (s, 1H), 3.86 (t, 4H) and 2.84 (t, 4H); MS (ESI) (M+H)⁺ 514.

### Example 18

### N-(5-{2-[(2-Fluorophenyl)amino]-6-oxo-1,6-dihydro-4-pyrimidinyl}-1H-benzimidazol-2-yl)-2-thiophenecarboxamide (Ir)

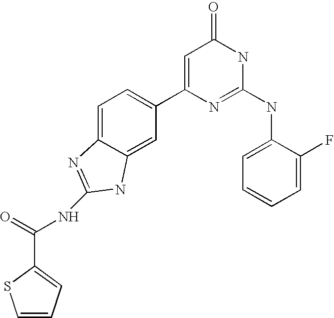

The title compound was prepared from N-{5-[6-(methyloxy)-2-(methylsulfonyl)-4-pyrimidinyl]-1H-benzimidazol-2-yl}-2-thiophenecarboxamide and 2-fluoroaniline as in example 5g. ¹H-NMR (400 MHz, d₆-DMSO) δ 12.43 (br, 2H), 11.07 (br, 1H), 8.78 (s, 1H), 8.50 (br, 1H), 8.15 (br, 1H), 8.11 (s, 1H), 7.95 (br, 1H), 7.82 (d, 1H), 7.48 (d, 1H), 7.35-7.28 (m, 2H), 7.22 (br, 1H), 7.18-7.12 (m, 1H) and 6.38 (s, 1H); MS (ESI) (M+H)⁺ 447.

### Example 19

### N-(5-{2-[(2,5-Dichlorophenyl)amino]-6-oxo-1,6-dihydro-4-pyrimidinyl}-1H-benzimidazol-2-yl)-2-thiophenecarboxamide (Is)

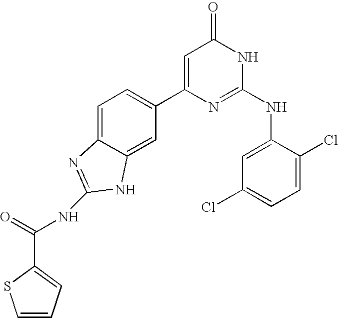

The title compound was prepared from N-{5-[6-(methyloxy)-2-(methylsulfonyl)-4-pyrimidinyl]-1H-benzimidazol-2-yl}-2-thiophenecarboxamide and 2,5-dichloroaniline as in example 5g. ¹H-NMR (400 MHz, d₆-DMSO) δ 12.40 (br, 2H), 8.77 (s, 1H), 8.45 (br, 1H), 8.04 (s, 1H), 7.86 (br, 1H), 7.80 (d, 1H), 7.56 (d, 1H), 7.48 (d, 1H), 7.20 (d, 1H), 7.18 (d, 1H) and 6.39 (br, 1H), interchangeable 2H could not be detected; MS (ESI) (M+H)⁺ 497.

### Example 20

### N-{5-[2-(Ethylamino)-6-oxo-1,6-dihydro-4-pyrimidinyl]-1H-benzimidazol-2-yl}-2-thiophenecarboxamide (It)

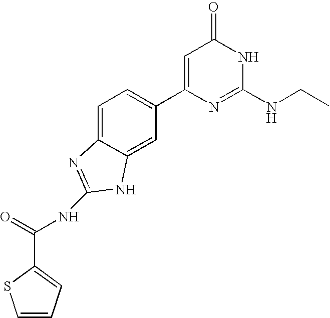

The title compound was prepared from N-{5-[6-(methyloxy)-2-(methylsulfonyl)-4-pyrimidinyl]-1H-benzimidazol-2-yl}-2-thiophenecarboxamide and aminoethane as in example 5g. ¹H-NMR (400 MHz, d₆-DMSO) δ 12.40 (br, 2H), 10.69 (br, 1H), 8.11 (s, 1H), 8.01 (br, 1H), 7.83 (br, 1H), 7.80 (dd, 1H), 7.44 (d, 1H), 7.20 (t, 1H), 6.50 (br, 1H), 6.08 (s, 1H), 3.43 (t, 2H) and 1.20 (t, 3H); MS (ESI) (M+H)⁺ 381.

### Example 21

### N-(5-{2-[(2-Methylphenyl)amino]-6-oxo-1,6-dihydro-4-pyrimidinyl}-1H-benzimidazol-2-yl)-2-thiophenecarboxamide (Iu)

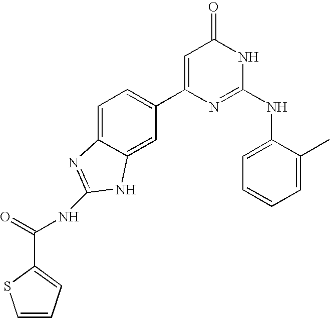

The title compound was prepared from N-{5-[6-(methyloxy)-2-(methylsulfonyl)-4-pyrimidinyl]-1H-benzimidazol-2-yl}-2-thiophenecarboxamide and o-toluidine as in example 5g. ¹H-NMR (400 MHz, d₆-DMSO) δ 12.38 (br, 2H), 8.21 (br, 1H), 8.05 (br, 3H), 7.86 (br, 1H), 7.77 (dd, 1H), 7.45 (d, 1H), 7.30 (d, 1H), 7.27 (d, 1H), 7.27 (t, 1H), 6.31 (s, 1H) and 2.30 (s, 3H), interchangeable 2H could not be detected; MS (ESI) (M+H)⁺ 443.

### Example 22

### N-[5-(6-Oxo-2-{[3-(2-oxo-1-pyrrolidinyl)propyl]amino}-1,6-dihydro-4-pyrimidinyl)-1H-benzimidazol-2-yl]-2-thiophenecarboxamide (Iv)

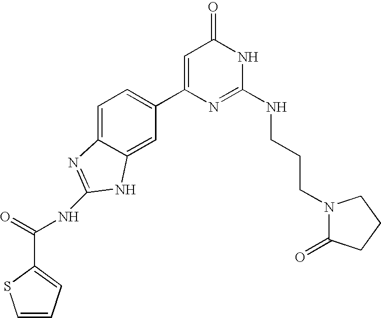

The title compound was prepared from N-{5-[6-(methyloxy)-2-(methylsulfonyl)-4-pyrimidinyl]-1H-benzimidazol-2-yl}-2-thiophenecarboxamide and 1-(3-aminopropyl)-2-pyrrolidinone as in example 5g. ¹H-NMR (400 MHz, d₆-DMSO) δ 12.33 (br, 2H), 8.15 (s, 1H), 8.08 (br, 1H), 7.83 (br, 1H), 7.80 (d, 1H), 7.43 (d, 1H), 7.19 (t, 1H), 6.08 (s, 1H), 2.23 (t, 2H), 1.92 (dt, 2H) and 1.78 (t, 2H), interchangeable 2H could not be detected, 6H were overlapped with water; MS (ESI) (M+H)⁺ 478.

### Example 23

### N-(5-{2-[(1,3-Dioxolan-2-ylmethyl)amino]-6-oxo-1,6-dihydro-4-pyrimidinyl}-1H-benzimidazol-2-yl)-2-thiophenecarboxamide (Iw)

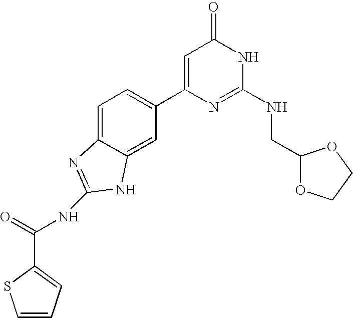

The title compound was prepared from N-{5-[6-(methyloxy)-2-(methylsulfonyl)-4-pyrimidinyl]-1H-benzimidazol-2-yl}-2-thiophenecarboxamide and 1-(1,3-dioxolan-2-yl)methylamine as in example 5g. ¹H-NMR (400 MHz, d₆-DMSO) δ 12.33 (br, 1H), 10.68 (br, 1H), 8.07 (s, 1H), 7.77 (d, 2H), 7.40 (d, 1H), 7.18 (s, 1H), 6.54 (br, 1H), 6.11 (s, 1H), 5.70 (t, 1H), 3.98 (m, 2H), 3.86 (m, 2H) and 3.64 (t, 2H), interchangeable 2H could not be detected; MS (ESI) (M+H)⁺ 439.

### Example 24

### 3-(Acetylamino)-N-(5-{2-[(2-chlorophenyl)amino]-6-oxo-1,6-dihydro-4-pyrimidinyl}-1H-benzimidazol-2-yl)benzamide (Ix)

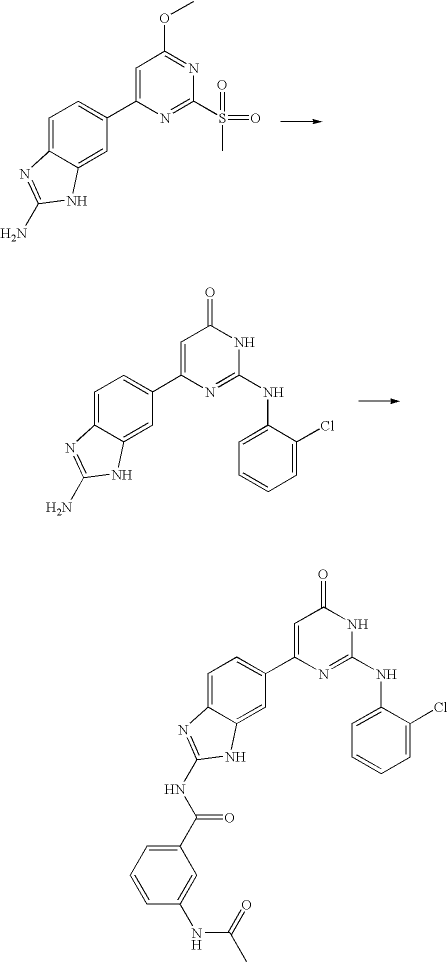

### a. 6-(2-Amino-1H-benzimidazol-5-yl)-2-[(2-chlorophenyl)amino]-4(1H)-pyrimidinone

5-[6-(Methyloxy)-2-(methylsulfonyl)-4-pyrimidinyl]-1H-benzimidazol-2-amine (320 mg, 1.0 mmol) and o-cholroaniline (1.0 mL, 10 mmol) were dissolved in NMP (3 mL) and HCl (4 N in dioxane, 1 mL). The reaction vessel was sealed and heated in a microwave reactor to 170° C. for 15 minutes. After cooled to ambient temperature, the mixture was neutralized with sat. NaHCO₃ aq. Water was poured into the mixture and the precipitated solid was collected by filtration, washed with CH₂Cl₂, and dried under vacuum to give the title compound (266.5 mg, 76%). ¹H-NMR (400 MHz, d₆-DMSO) δ 11.0 (br, 1H), 8.47 (d, 1H), 8.40 (s, 1H), 7.73 (s, 1H), 7.55 (d, 1H), 7.44 (t, 1H), 7.17 (m, 3H), 6.35 (br, 2H), 6.30 (br, 2H) and 6.30 (br, 1H); MS (ESI) (M+H)⁺ 353.

### b. 3-(Acetylamino)-N-(5-{2-[(2-chlorophenyl)amino]-6-oxo-1,6-dihydro-4-pyrimidinyl}-1H-benzimidazol-2-yl)benzamide

The title compound was prepared from 6-(2-amino-1H-benzimidazol-5-yl)-2-[(2-chlorophenyl)amino]-4(1H)-pyrimidinone and 3-(acetylamino)benzoic acid as in example 5f. ¹H-NMR (400 MHz, d₆-DMSO) δ 12.36 (br, 2H), 10.16 (s, 1H), 8.50 (d, 2H), 8.29 (s, 1H), 8.12 (s, 1H), 7.81 (m, 3H), 7.54 (dd, 1H), 7.49 (d, 1H), 7.47-7.42 (m, 2H), 7.16 (dt, 1H), 6.37 (s, 1H) and 2.08 (s, 3H); MS (ESI) (M+H)⁺ 514.

### Example 25

### N-(5-{2-[(2-Chlorophenyl)amino]-6-oxo-1,6-dihydro-4-pyrimidinyl}-1H-benzimidazol-2-yl)cyclopropanecarboxamide (Iy)

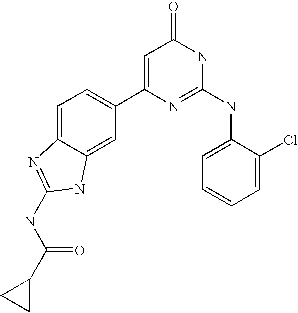

The title compound was prepared from 6-(2-amino-1H-benzimidazol-5-yl)-2-[(2-chlorophenyl)amino]-4(1H)-pyrimidinone and cyclopropanecarboxylic acid as in example 5f. ¹H-NMR (400 MHz, d₆-DMSO) δ 12.17 (br, 1H), 11.91 (br, 1H), 8.48 (br, 2H), 8.08 (br, 1H), 7.73 (dd, 1H), 7.52 (d, 1H), 7.45 (br, 2H), 7.17 (t, 1H), 6.36 (br, 1H), 1.89 (br, 1H) and 0.93 (s, 4H), interchangeable 1H could not be detected; MS (ESI) (M+H)⁺ 421.

### Example 26

### 4-(Aminosulfonyl)-N-(5-{2-[(2-chlorophenyl)amino]-6-oxo-1,6-dihydro-4-pyrimidinyl}-1H-benzimidazol-2-yl)benzamide (Iz)

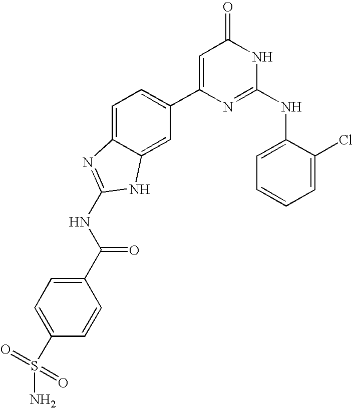

The title compound was prepared from 6-(2-amino-1H-benzimidazol-5-yl)-2-[(2-chlorophenyl)amino]-4(1H)-pyrimidinone and 4-(aminosulfonyl)benzoic acid as in example 5f. ¹H-NMR (400 MHz, d₆-DMSO) δ 12.54 (br, 2H), 8.50 (br, 2H), 8.28 (d, 2H), 8.11 (s, 1H), 7.95 (d, 2H), 7.82 (dd, 1H), 7.55 (dd, 1H), 7.52-7.44 (m, 4H), 7.17 (dt, 1H) and 6.40 (s, 1H), interchangeable 1H could not be detected; MS (ESI) (M+H)⁺ 536.

### Example 27

### N-(5-{2-[(2-Chlorophenyl)amino]-6-oxo-1,6-dihydro-4-pyrimidinyl}-1H-benzimidazol-2-yl)-4-[(dipropylamino)sulfonyl]benzamide (Iaa)

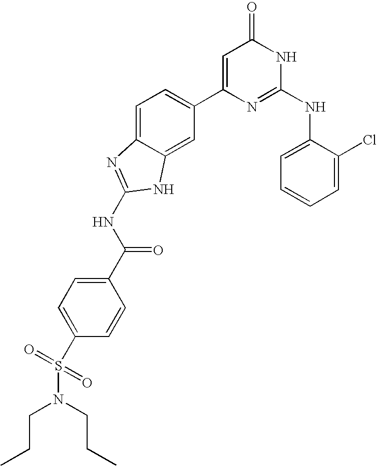

The title compound was prepared from 6-(2-amino-1H-benzimidazol-5-yl)-2-[(2-chlorophenyl)amino]-4(1H)-pyrimidinone and 4-[(dipropylamino)sulfonyl]benzoic acid as in example 5f. ¹H-NMR (400 MHz, d₆-DMSO) δ 12.54 (br, 2H), 8.05 (br, 1H), 8.48 (d, 1H), 8.31 (d, 2H), 8.11 (s, 1H), 7.82 (dd, 1H), 7.93 (d, 1H), 7.84 (dd, 1H), 7.55 (dd, 1H), 7.50 (d, 1H), 7.47 (t, 1H), 7.18 (dt, 1H), 6.41 (s, 1H), 3.07 (t, 4H), 1.48 (tq, 4H) and 0.82 (t, 6H), interchangeable 1H could not be detected; MS (ESI) (M+H)⁺ 620.

### Example 28

### N-(5-{2-[(2-Chlorophenyl)amino]-6-oxo-1,6-dihydro-4-pyrimidinyl}-1H-benzimidazol-2-yl)-1H-imidazole-4-carboxamide (Iab)

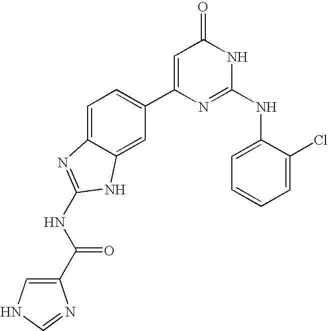

The title compound was prepared from 6-(2-amino-1H-benzimidazol-5-yl)-2-[(2-chlorophenyl)amino]-4(1H)-pyrimidinone and 1H-imidazole-4-carboxylic acid as in example 5f. ¹H-NMR (400 MHz, d₆-DMSO) δ 8.48 (br, 1H), 8.12 (br, 1H), 8.08 (s, 1H), 7.89 (s, 1H), 7.77 (dd, 1H), 7.55 (dd, 1H), 7.47 (br, 1H), 7.46 (d, 1H), 7.16 (dt, 1H), 6.33 (br, 1H) and 6.37 (s, 1H), interchangeable 4H could not be detected; MS (ESI) (M+H)⁺ 447.

### Example 29

### N-(5-{2-[(2-Chlorophenyl)amino]-6-oxo-1,6-dihydro-4-pyrimidinyl}-1H-benzimidazol-2-yl)-3,4,5-tris(methyloxy)benzamide (Iac)

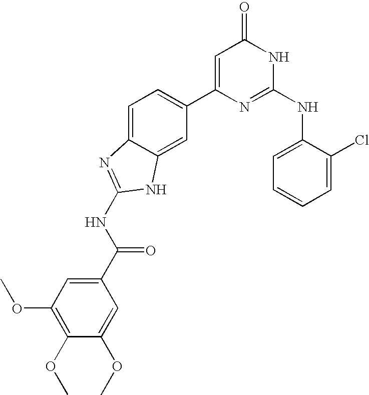

The title compound was prepared from 6-(2-amino-1H-benzimidazol-5-yl)-2-[(2-chlorophenyl)amino]-4(1H)-pyrimidinone and 3,4,5-trimethoxybenzoic acid as in example 5f. ¹H-NMR (400 MHz, d₆-DMSO) δ 12.33 (br, 2H), 8.50 (br, 2H), 8.13 (s, 1H), 7.79 (d, 1H), 7.54 (dd, 1H), 7.51 (s, 2H), 7.50 (s, 1H), 7.45 (t, 1H), 7.15 (t, 1H), 6.38 (s, 1H), 3.89 (s, 6H) and 3.75 (s, 3H), interchangeable 1H could not be detected; MS (ESI) (M+H)⁺ 547.

### Example 30

### N-(5-{2-[(2-Chlorophenyl)amino]-6-oxo-1,6-dihydro-4-pyrimidinyl}-1H-benzimidazol-2-yl)-3-pyridinecarboxamide (Iad)

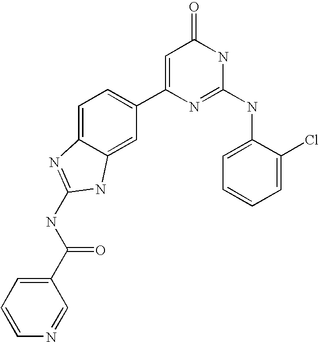

The title compound was prepared from 6-(2-amino-1H-benzimidazol-5-yl)-2-[(2-chlorophenyl)amino]-4(1H)-pyrimidinone and 3-pyridinecarboxylic acid as in example 5f. ¹H-NMR (400 MHz, d₆-DMSO) δ 9.27 (s, 1H), 8.76 (d, 1H), 8.48 (d, 1H), 8.46 (t, 1H), 8.44 (t, 1H), 8.12 (s, 1H), 7.83 (d, 1H), 7.57 (d, 1H), 7.51-7.45 (m, 2H), 7.18 (t, 1H) and 6.41 (s, 1H), interchangeable 4H could not be detected; MS (ESI) (M+H)⁺ 458.

### Example 31

### N-(5-{2-[(2-chlorophenyl)amino]-6-oxo-1,6-dihydro-4-pyrimidinyl}-1H-benzimidazol-2-yl)-2-(methyloxy)acetamide (Iae)

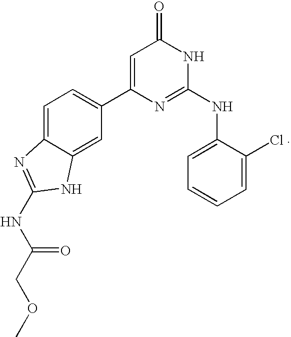

The title compound was prepared from 6-(2-amino-1H-benzimidazol-5-yl)-2-[(2-chlorophenyl)amino]-4(1H)-pyrimidinone and methoxyacetic acid as in example 5f. ¹H-NMR (400 MHz, d₆-DMSO) δ 12.24 (br, 1H), 11.48 (br, 2H), 8.49 (br, 2H), 8.10 (br, 1H), 7.75 (dd, 1H), 7.53 (dd, 1H), 7.45 (m, 2H), 7.15 (t, 1H), 6.36 (br, 1H), 4.16 (s, 2H) and 3.39 (s, 3H); MS (ESI) (M+H)⁺ 425.

### Example 32

### N-(5-{2-[(2-Chlorophenyl)amino]-6-oxo-1,6-dihydro-4-pyrimidinyl}-1H-benzimidazol-2-yl)-2-(4-methyl-1-piperazinyl)acetamide (Iaf)

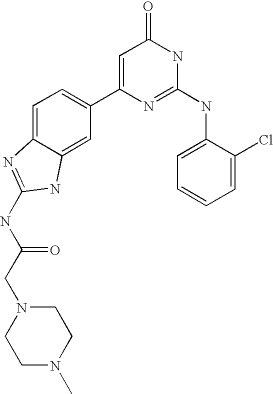

The title compound was prepared from 6-(2-amino-1H-benzimidazol-5-yl)-2-[(2-chlorophenyl)amino]-4(H)-pyrimidinone and (4-methyl-1-piperazinyl)acetic acid as in example 5f. ¹H-NMR (400 MHz, d₆-DMSO) δ 12.27 (br, 1H), 11.22 (br, 2H), 8.49 (br, 2H), 8.10 (br, 1H), 7.76 (dd, 1H), 7.54 (dd, 1H), 7.45 (br, 2H), 7.15 (t, 1H), 6.36 (br, 1H), 3.29 (s, 2H), 2.57 (br, 4H), 2.36 (br, 4H) and 2.16 (s, 3H); MS (ESI) (M+H)⁺ 493.

### Example 33

### 4-(4-Methyl-1-piperazinyl)-N-{5-[6-oxo-2-(phenylamino)-1,6-dihydro-4-pyrimidinyl]-1H-benzimidazol-2-yl}benzamide (Iag)

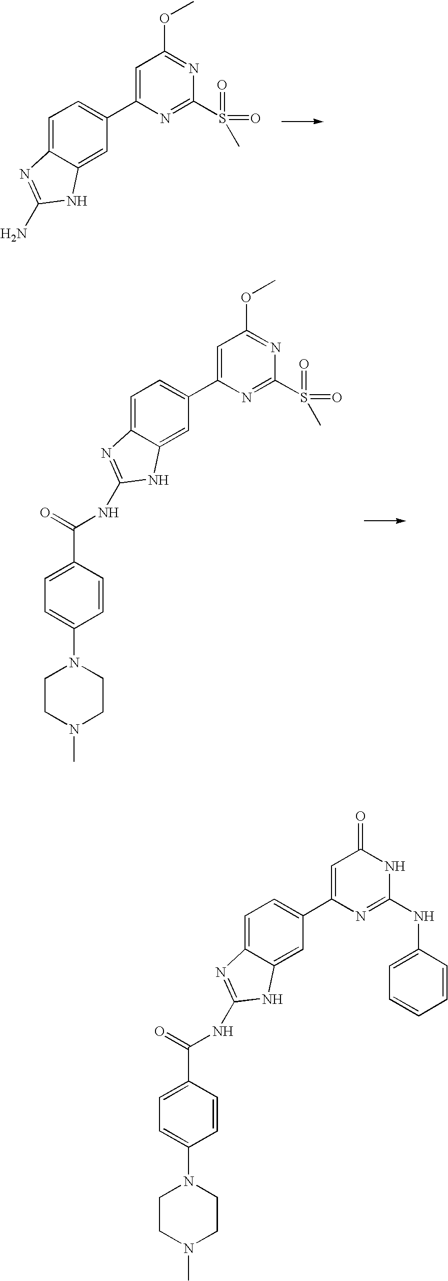

### a. N-{5-[6-(methyloxy)-2-(methylsulfonyl)-4-pyrimidinyl]-1H-benzimidazol-2-yl}-4-(4-methyl-1-piperazinyl)benzamide

The title compound was prepared from 5-[6-(methyloxy)-2-(methylsulfonyl)-4-pyrimidinyl]-1H-benzimidazol-2-amine and 4-(4-methyl-1-piperazinyl)benzoic acid as in example 5f; MS (ESI) (M+H)⁺ 522.

### b. 4-(4-Methyl-1-piperazinyl)-N-{5-[6-oxo-2-(phenylamino)-1,6-dihydro-4-pyrimidinyl]-1H-benzimidazol-2-yl}benzamide

The title compound was prepared from N-{5-[6-(methyloxy)-2-(methylsulfonyl)-4-pyrimidinyl]-1H-benzimidazol-2-yl}-4-(4-methyl-1-piperazinyl)benzamide and aniline as in example 5g. ¹H-NMR (400 MHz, d₆-DMSO) δ 8.18 (s, 1H), 8.05 (d, 2H), 7.88 (d, 2H), 7.81 (d, 1H), 7.49 (d, 1H), 7.37 (t, 3H), 7.02 (m, 4H), 6.30 (s, 1H), 2.43 (br, 8H) and 2.22 (s, 3H), interchangeable 2H could not be detected; MS (ESI) (M+H)⁺ 521.

### Example 34

### 6-(2-Amino-1H-benzimidazol-5-yl)-2-({[3-(methyloxy)phenyl]methyl}amino)-4(1H)-pyrimidinone (Iah)

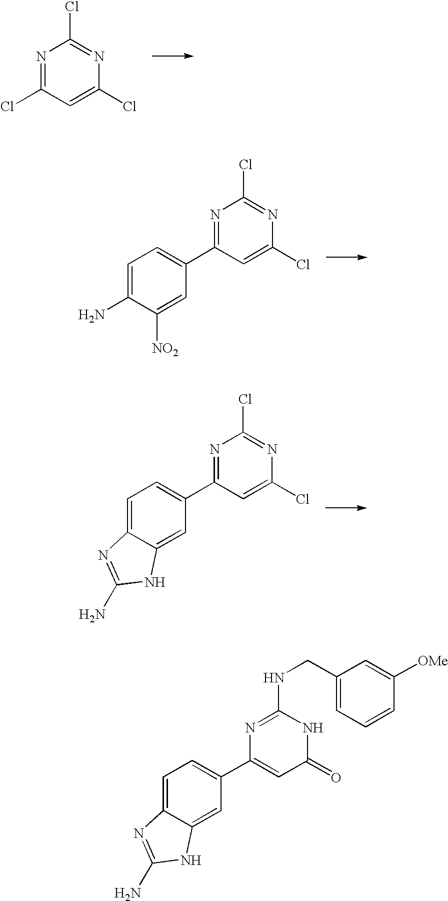

### a. 4-(2,6-Dichloro-4-pyrimidinyl)-2-nitroaniline

The title compound was prepared from 2,4,6-trichlorotriazine and [2-nitro-4-(4,4,5,5-tetramethyl-1,3,2-dioxaborolan-2-yl)aniline as described in example 1a; MS (ESI) (M+H)⁺ 285.

### b. 5-(2,6-Dichloro-4-pyrimidinyl)-1H-benzimidazol-2-amine

To a solution of 4-(2,6-dichloro-4-pyrimidinyl)-2-nitroaniline (285.1 mg. 10 mmol) in AcOH (10 mL), Zn powder (ca 400 mg) was added and stirred at room temperature for 1.5 hrs. The mixture was diluted with MeOH then insoluble materials were removed by filtration. After evaporation, EtOH (40 mL) was added to the residue and followed by BrCN (530 mg, 5.0 mmol), then stirred at room temperature 2 hrs. After evaporation, the mixture was purified on NH₂-SPE and recrystallized from CH₂Cl₂-MeOH to give the title compound (108.3 mg, 39%); MS (ESI) (M+H)⁺ 280.

### c. 6-(2-Amino-1H-benzimidazol-5-yl)-2-({[3-(methyloxy)phenyl]methyl}amino) 4(1H)-pyrimidinone

The title compound was prepared from 5-(2,6-dichloro-4-pyrimidinyl)-1H-benzimidazol-2-amine and 3-methoxybenzylamine as described in example 3.

¹H-NMR (400 MHz, d₆-DMSO) δ 10.70(br, 1H), 7.78(s, 1H), 7.58(dd, 1H), 7.27(dd, 1H), 7.09(d, 1H), 7.00-6.95(2H), 6.94(br, 1H), 6.83(d, 1H) 6.32(br, 2H), 6.04(s, 1H), 4.58(d, 2H) and 3.73(s, 3H), interchangeable 1H could not be detected; MS (ESI) (M+H)⁺ 363.

### Example 35

### N-{5-[2-({[3-(Methyloxy)phenyl]methyl}amino)-6-oxo-1,6-dihydro-4-pyrimidinyl]-1H-benzimidazol-2-yl}-2-thiophenecarboxamide (Iai)

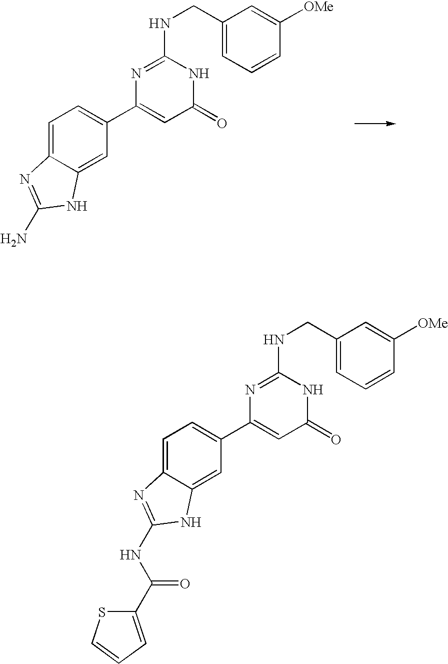

The title compound was prepared from 6-(2-amino-1H-benzimidazol-5-yl)-2-({[3-(methyloxy)phenyl]methyl}amino)-4(1H)-pyrimidin one and 2-thiophenecarboxylic acid as described in example 5f. ¹H-NMR (400 MHz, d₆-DMSO) δ 8.14(s, 1H), 8.01(br, 1H), 7.86-7.78(2H), 7.43(d, 1H), 7.27(dd, 1H), 7.21(d, 1H), 7.10-6.98(3H), 6.84(d, 1H), 6.13(s, 1H), 4.60(d, 2H) and 3.73(s, 3H), interchangeable 3H could not be detected; MS (ESI) (M+H)⁺ 473.

### Example 36

### 6-Benzo[1,3]dioxol-5-yl-2-(3-methoxy-benzylamino)-1H-pyrimidin-4-one (Iaj)

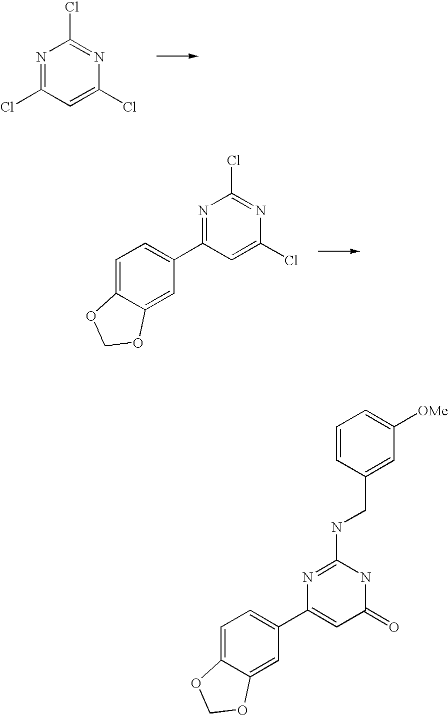

### a. 4-Benzo[1,3]dioxol-5-yl-2,6-dichloro-pyrimidine

The title compound was prepared from 2,4,6-trichlorotriazine and 3,4-methylenedioxybenzene boronic acid as described in example 1a. MS (ESI) (M+H)⁺ 269.

### b. 6-Benzo[1,3]dioxol-5-yl-2-(3-methoxy-benzylamino)-1H-pyrimidin-4-one

The title compound was prepared from 4-benzo[1,3]dioxol-5-yl-2,6-dichloro-pyrimidine and 3-methoxybenzylamine as described in example 3.

¹H-NMR (400 MHz, d₆-DMSO) δ 10.70(br, 1H), 7.58(dd, 1H), 7.52(s, 1H), 7.25(dd, 1H), 7.06(br, 1H), 6.97-6.86(3H), 6.82(dd, 1H), 6.09(s, 1H) 6.07(s, 2H), 4.54(d, 2H) and 3.73(s, 3H); MS (ESI) (M+H)⁺ 352.

### Example 37

### 6-(1,3-Benzodioxol-5-yl)-2-(1H-indazol-5-ylamino)-4(1H)-pyrimidinone (Iak)

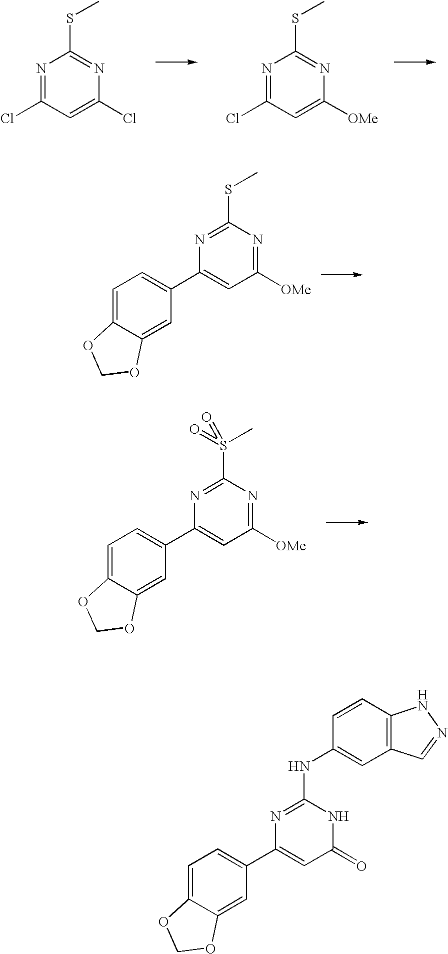

### a. 4-Chloro-6-(methyloxy)-2-(methylthio)pyrimidine

To a solution of 4,6-dichloro-2-(methylthio)pyrimidine (7.8 g, 40 mmol) in MeOH (100 mL), NaOMe (4.1 M in MeOH, 10.2 mL) was added at 35° C. and stirred at same temperature for 1 hr. After evaporation, the mixture was extracted with CH₂Cl₂ and washed with water. The organic layer was dried over Na₂SO₄ then evaporated. The residue was purified on SiO₂ chromatography to give the title compound (5.8 g, 75%); MS (ESI) (M+H)⁺ 192.

### b. 4-(1,3-Benzodioxol-5-yl)-6-(methyloxy)-2-(methylthio)pyrimidine

The title compound was prepared from 4-chloro-6-(methyloxy)-2-(methylthio)pyrimidine and 3,4-methylenedioxybenzene boronic acid as described in example 1a; MS (ESI) (M+H)⁺ 277.

### c. 4-(1,3-Benzodioxol-5-yl)-6-(methyloxy)-2-(methylsulfonyl)pyrimidine

To a solution of 4-(1,3-benzodioxol-5-yl)-6-(methyloxy)-2-(methylthio)pyrimidine (27.6 mg, 0.1 mmol) in CH₂Cl₂ (2 mL), mCPBA (80% assay, 45.3 mg, 0.21 mmol) was added at 0° C. then stirred at room temperature. After 30 min, mCPBA (30 mg, 0.14 mmol) was added and stirred at room temperature for 1 hr. The mixture was purified on SCX-SPE then NH₂-SPE to give the title compound (27.1 mg, 88%); MS (ESI) (M+H)⁺ 309.

### d. 6-(1,3-Benzodioxol-5-yl)-2-(1H-indazol-5-ylamino)-4(1H)-pyrimidinone

A mixture of 4-(1,3-benzodioxol-5-yl)-6-(methyloxy)-2-(methylsulfonyl)pyrimidine (27.1 mg, 0.088 mmol), 1H-indazol-5-amine (23 mg, 0.18 mmol) and 4M HCl-dioxane (0.4 mL) was heated by irradiation of microwave at 150° C. for 6 hrs. After cooling, the mixture was purified on SCX-SPE then on NH₂-SPE to give the title compound (yield not determined).

¹H-NMR (400 MHz, d₆-DMSO) δ 13.02(br, 1H), 10.73(br, 1H), 8.82(br, 1H), 8.08(br, 1H), 8.04(s, 1H), 7.62(d, 1H), 7.58-7.49(3H), 7.00(d, 1H), 6.30(br, 1H) and 6.09(s, 2H); MS (ESI) (M+H)⁺ 348.

### Example 38

### 6-(1,3-Benzodioxol-5-yl)-2-{[3-(methyloxy)phenyl]amino}-4(1H)-pyrimidinone (Ial)

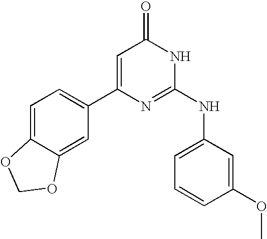

The title compound was prepared from 4-(1,3-benzodioxol-5-yl)-6-(methyloxy)-2-(methylsulfonyl)pyrimidine and m-anisidine as in example 37d. ¹H-NMR (400 MHz, d₆-DMSO) δ 7.64 (d, 1H), 7.62 (s, 1H), 7.25 (t, 1H), 7.10 (br, 1H), 7.02 (d, 1H), 6.63 (d, 1H), 6.37 (s, 1H), 6.10 (s, 2H) and 3.79 (s, 3H), interchangeable 3H could not be detected; MS (ESI) (M+H)⁺ 339.

### Example 39

### 3-{[4-(1,3-Benzodioxol-5-yl)-6-oxo-1,6-dihydro-2-pyrimidinyl]amino}benzenesulfonamide (Iam)

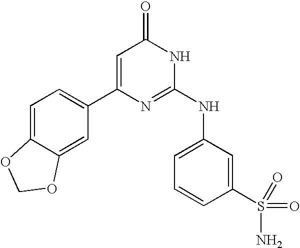

The title compound was prepared from 4-(1,3-benzodioxol-5-yl)-6-(methyloxy)-2-(methylsulfonyl)pyrimidine and 3-aminobenzenesulfonamide as in example 37d. ¹H-NMR (400 MHz, d₆-DMSO) δ 8.50 (br, 1H), 7.78 (br, 1H), 7.71 (d, 1H), 7.62 (s, 1H), 7.54 (t, 1H), 7.47 (d, 1H), 7.34 (br, 2H), 6.97 (d, 1H), 6.44 (br, 1H), 6.09 (s, 2H), interchangeable 2H could not be detected; MS (ESI) (M+H)⁺ 387.

### Example 40

### 6-(1,3-Benzodioxol-5-yl)-2-{[2-(methyloxy)phenyl]amino}-4(1H)-pyrimidinone (Ian)

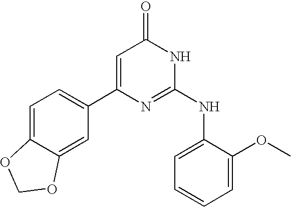

The title compound was prepared from 4-(1,3-benzodioxol-5-yl)-6-(methyloxy)-2-(methylsulfonyl)pyrimidine and o-anisidine as in example 38d. ¹H-NMR (400 MHz, d₆-DMSO) δ 8.46 (d, 2H), 7.62 (d, 1H), 7.55 (s, 1H), 7.11-6.99 (m, 4H), 6.31 (s, 1H), 6.10 (s, 2H) and 3.90 (s, 3H), interchangeable 1H could not be detected; MS (ESI) (M+H)⁺ 339.

### Example 41

### 2-[(2-Chlorophenyl)amino]-6-(4-pyridinyl)-4(1H)-pyrimidinone (Io)

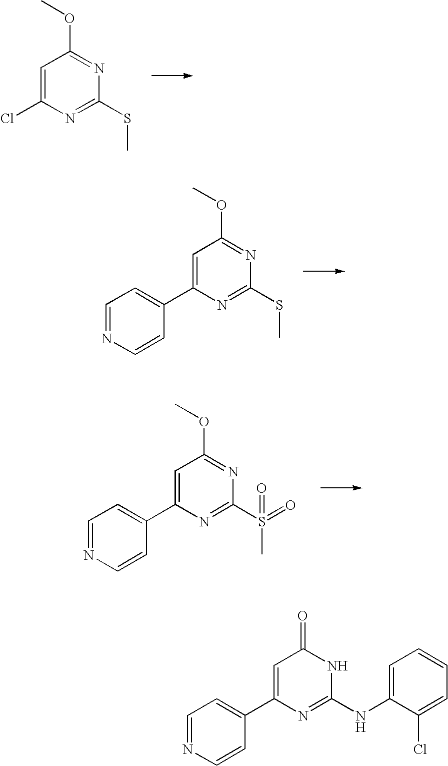

### a. 4-(Methyloxy)-2-(methylthio)-6-(4-pyridinyl)pyrimidine

To a mixture of 4-chloro-6-(methyloxy)-2-(methylthio)pyrimidine (2.3 g, 10 mmol), 4-pyridinylboronic acid (1.47 g, 12 mmol) and Pd(PPh₃)₄ (577 mg, 5 mol %), DMF (100 mL) and 2M Cs₂CO₃ aq. (20 mL) were added and stirred at 100° C. overnight. The mixture was extracted with CH₂Cl₂ then the organic layer was washed with water. After drying over Na₂SO₄ and evaporation, the mixture was washed with EtOAc to give the title compound (1.95 g, 84%); MS (ESI) (M+H)⁺ 234.

### b. 4-(Methyloxy)-2-(methylsulfonyl)-6-(4-pyridinyl)pyrimidine

The title compound was prepared from 4-(methyloxy)-2-(methylthio)-6-(4-pyridinyl)pyrimidine as in example 5e; MS (ESI) (M+H)^{+ 266.}

### c. 2-[(2-Chlorophenyl)amino]-6-(4-pyridinyl)-4(1H)-pyrimidinone

The title compound was prepared from 4-(methyloxy)-2-(methylsulfonyl)-6-(4-pyridinyl)pyrimidine and o-chlorlaniline as in example 5g. ¹H-NMR (400 MHz, d₆-DMSO) δ 8.66 (d, 2.08), 8.31 (d, 1H), 7.87 (dd, 2H), 7.52 (d, 1H), 7.39 (t, 1H), 7.13 (t, 1H) and 6.54 (s, 1H), interchangeable 2H could not be detected; MS (ESI) (M+H)⁺ 299.

### Example 42

### 2-(1H-Indazol-6-ylamino)-6-(4-pyridinyl)-4(1H)-pyrimidinone (Iap)

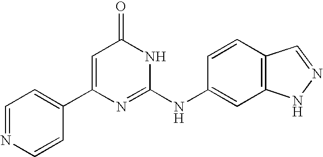

The title compound was prepared from 4-(methyloxy)-2-(methylsulfonyl)-6-(4-pyridinyl)pyrimidine and 1H-indazol-6-amine as in example 5g. ¹H-NMR (400 MHz, d₆-DMSO) δ 12.98 (s, 1H), 8.73 (d, 2H), 8.35 (br, 1H), 8.02 (dd, 1H), 7.99 (s, 1H), 7.70 (d, 1H), 7.17 (br, 1H) and 6.65 (s, 1H), interchangeable 3H could not be detected; MS (ESI) (M+H)⁺ 305.

### Example 43

### 3-{[4-Oxo-6-(4-pyridinyl)-1,4-dihydro-2-pyrimidinyl]amino}benzenesulfonamide (Iaq)

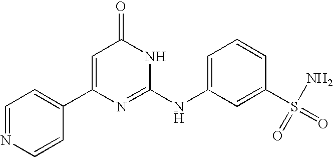

The title compound was prepared from 4-(methyloxy)-2-(methylsulfonyl)-6-(4-pyridinyl)pyrimidine and 3-aminobenzenesulfonamide as in example 5g. ¹H-NMR (400 MHz, d₆-DMSO) δ 8.82 (d, 2H), 8.77 (br,1H), 8.19 (d, 2H), 7.91 (br, 1H), 7.70 (t, 1H), 7.63 (d, 1H), 7.52 (s, 1H) and 6.84 (br, 1H), interchangeable 3H could not be detected; MS (ESI) (M+H)⁺ 344.

### Example 44

### 2-{[3-(methyloxy)phenyl]amino}-6-(4-pyridinyl)-4(1H)-pyrimidinone (Iar)

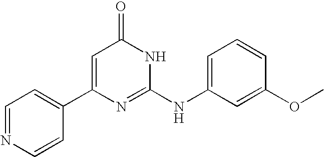

The title compound was prepared from 4-(methyloxy)-2-(methylsulfonyl)-6-(4-pyridinyl)pyrimidine and 3-(methyloxy)aniline as in example 5g. ¹H-NMR (400 MHz, d₆-DMSO) δ 11.48 (br, 1H), 8.72 (d, 2H), 8.54 (br, 1H), 8.47 (d, 1H), 7.95 (d, 2H), 7.09-7.03 (m, 3H), 6.58 (s, 1H) and 3.91 (s, 3H); MS (ESI) (M+H)⁺ 295.

### Example 45

### 2-(Phenylamino)-6-(4-pyridinyl)-4(1H)-pyrimidinone (Ias)

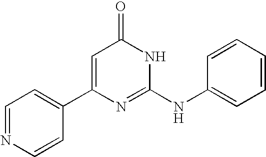

The title compound was prepared from 4-(methyloxy)-2-(methylsulfonyl)-6-(4-pyridinyl)pyrimidine and aniline as in example 5g. ¹H-NMR (400 MHz, d₆-DMSO) δ 11.00 (br, 1H), 9.02 (br, 1H), 8.71 (d, 2H), 7.95 (d, 2H), 7.71 (d, 2H), 7.38 (t, 2H), 7.07 (t, 1H) and 6.61 (s, 1H); MS (ESI) (M+H)⁺ 265.

### Example 46

### Ethyl 3-{[4-oxo-6-(4-pyridinyl)-1,4-dihydro-2-pyrimidinyl]amino}benzoate (Iat)

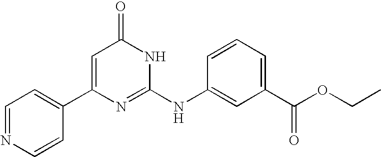

The title compound was prepared from 4-(methyloxy)-2-(methylsulfonyl)-6-(4-pyridinyl)pyrimidine and ethyl 3-aminobenzoate as in example 5g. ¹H-NMR (400 MHz, d₆-DMSO) δ 11.18 (br, 1H), 9.38 (br, 1H), 8.72 (d, 2H), 8.66 (br, 1H), 8.02 (d, 2H), 7.83 (brd, 1H), 7.66 (d, 1H), 7.51 (dd, 1H), 6.69 (br, 1H), 4.37 (q, 2H) and 1.33 (t, 3H); MS (ESI) (M+H)⁺ 337.

### Example 47

### 4-{[4-Oxo-6-(4-pyridinyl)-1,4-dihydro-2-pyrimidinyl]amino}benzenesulfonamide (Iau)

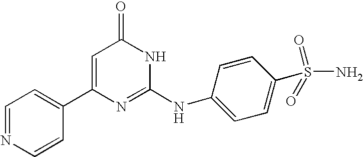

The title compound was prepared from 4-(methyloxy)-2-(methylsulfonyl)-6-(4-pyridinyl)pyrimidine and 4-aminobenzenesulfonamide as in example 5g. ¹H-NMR (400 MHz, d₆-DMSO) δ 8.73 (d, 2H), 8.00 (d, 2H), 7.93 (br, 2H), 7.81 (d, 2H), 7.25 (s, 2H) and 6.72 (br, 1H), interchangeable 2H could not be detected; MS (ESI) (M+H)⁺ 344.

### Example 48

### 6-(4-Pyridinyl)-2-(4-pyridinylamino)-4(1H)-pyrimidinone (Iav)

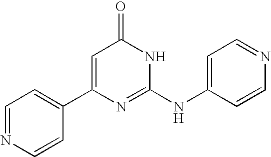

The title compound was prepared from 4-(methyloxy)-2-(methylsulfonyl)-6-(4-pyridinyl)pyrimidine and 4-pyridinamine as in example 5g. ¹H-NMR (400 MHz, d₆-DMSO) δ 9.32 (d, 2H), 8.66 (d, 2H), 8.54 (s, 2H), 8.03 (d, 2H), 6.93 (d, 2H) and 6.62 (s, 1H); MS (ESI) (M+H)⁺ 266.

### Example 49

### N-(3-{[4-Oxo-6-(4-pyridinyl)-1,4-dihydro-2-pyrimidinyl]amino}phenyl)acetamide (Iaw)

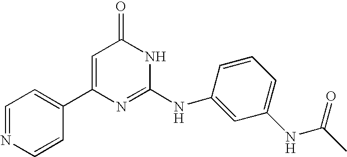

The title compound was prepared from 4-(methyloxy)-2-(methyslsulfonyl)-6-(4-pyridinyl)pyrimidine and N-(3-aminophenyl)acetamide as in example 5g. ¹H-NMR (400 MHz, d₆-DMSO) δ 9.97 (br, 1H), 9.69 (dd, 2H), 8.21 (br, 1H), 8.05 (dd, 2H), 7.44 (d, 1H), 7.27 (dd, 1H), 7.10 (d, 1H), 6.59 (br, 1H) and 2.08 (s, 3H), interchangeable 3H could not be detected; MS (ESI) (M+H)+ 322.

Biological Methods and Data

As demonstrated by the representative compounds of the present invention in Table 1, the compounds of the present invention have valuable pharmacological properties due to their potent ability to inhibit the hYAK3 kinase enzyme.

Substrate phosphorylation assays were carried out as follows:

YAK3 Scintillation Proximity Assays Using Ser164 of Myelin Basic Protein as the phosphoacceptor

The source of Ser164 substrate oeptide The biotinylated Ser164, S164A peptide (Biotinyl-LGGRDSRAGS*PMARR-OH), sequence derived from the C-terminus of bovine myelin basic protein (MBP) with Ser162 substituted as Ala162, was purchased from California Peptide Research Inc. (Napa, Calif.), and its purity was determined by HPLC. Phosphorylation occurs at position 164 (marked S* above). The calculated molecular mass of the peptide was 2166 dalton. Solid sample was dissolved at 10 mM in DMSO, aliquoted, and stored at −20° C. until use.

The source of Enzyme:

hYAK3: Glutathione-S-Transferase (GST)-hYak3-His6 containing amino acid residues 124-526 of human YAK3 (aa 124-526 of SEQ ID NO 2. in U.S. Pat. No. 6,323,318) was purified from baculovirus expression system in Sf9 cells using Glutathione Sepharose 4B column chromatography followed by Ni-NTA-Agarose column chromatography. Purity greater than 65% typically was achieved. Samples, in 50 mM Tris, 150 mM NaCl, 10% glycerol, 0.1% Triton, 250 mM imidazole, 10 mM β-mercapto ethanol, pH 8.0. were stored at −80° C. until use.

Kinase assay of purified hYAK3: Assays were performed in 96 well (Costar, Catalog No. 3789) or 384 well plates (Costar, Catalog No. 3705). Reaction (in 20, 25, or 40 μl volume) mix contained in final concentrations 25 mM Hepes buffer, pH 7.4; 10 mM MgCl₂; 10 mM β-mercapto ethanol; 0.0025% Tween-20; 0.001 mMATP, 0.1 μCi of [γ-³³P]ATP; purified hYAK3 (7-14 ng/assay; 4 nM final); and 4 μM Ser164 peptide. Compounds, titrated in DMSO, were evaluated at concentrations ranging from 50 μM to 0.5 nM. Final assay concentrations of DMSO did not exceed 5%, resulting in less than 15% loss of YAK3 activity relative to controls without DMSO. Reactions were incubated for 2 hours at room temperature and were stopped by a 75 ul addition of 0.19 μg Streptavidin Scintillation Proximity beads (Amersham Pharmacia Biotech, Catalog No. RPNQ 0007) in PBS, pH 7.4, 10 mM EDTA, 0.1% Triton X-100, 1 mM ATP. Under the assay conditions defined above, the Kₘ(apparent) for ATP was determined to be 7.2+-2.4 μM.

TABLE 1

| Compound no. | pIC₅₀ values |
|---|---|
| Ig | +++ |
| Il | ++ |
| Ip | + |

Legend

| | |
|---|---|
| pIC₅₀ values | Symbol |
| 8.99-8 | +++ |
| 7.99-7 | ++ |
| 6.99-6 | + |

pIC₅₀ = −log₁₀(IC₅₀)

Utility of the Present Invention

The above biological data clearly shows that the compounds of formula I are useful for treating or preventing disease states in which hYAK3 proteins are implicated, especially diseases of the erythroid and hematopoietic systems, including but not limited to, anemias due to renal insufficiency or to chronic disease, such as autoimmunity, HIV, or cancer, and drug-induced anemias, myelodysplastic syndrome, aplastic anemia, myelosuppression, and cytopenia.

The compounds of formula I are especially useful in treating diseases of the hematopoietic system, particularly anemias. Such anemias include an anemia selected from the group comprising: aplastic anemia and myelodysplastic syndrome. Such anemias also include those wherein the anemia is a consequence of a primary disease selected from the group consisting of: cancer, leukemia and lymphoma. Such anemias also include those wherein the anemia is a consequence of a primary disease selected from the group consisting of: renal disease, failure or damage. Such anemias include those wherein the anemia is a consequence of chemotherapy or radiation therapy, in particular wherein the chemotherapy is chemotherapy for cancer or AZT treatment for HIV infection. Such anemias include those wherein the anemia is a consequence of a bone marrow transplant or a stem cell transplant. Such anemias also include anemia of newborn infants. Such anemias also include those which are a consequence of viral, fungal, microbial or parasitic infection.

The compounds of formula I are also useful for enhancing normal red blood cell numbers. Such enhancement is desirable for a variety of purposes, especially medical purposes such as preparation of a patient for transfusion and preparation of a patient for surgery.

What is claimed is:

1. A compound of the formula I, or a salt, solvate, or a physiologically functional derivative thereof 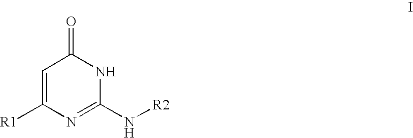
in which
R2 is a radical of the formula 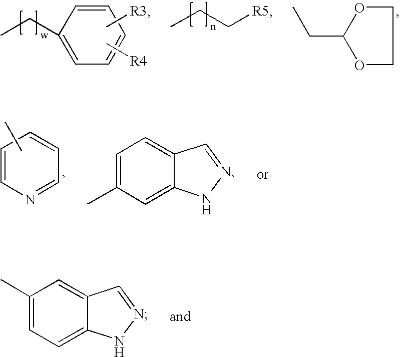
in which
n=1-2;
w=0-2;
R3 and R4 are independently hydrogen, hydroxy, —OR6, halogen, —SO₂NH₂, —OH, —C₁₋₆alkyl, —(C═O)—OEt, —NH(C═O)—CH₃, or a radical of the formula 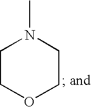
R5 is —NH₂, hydrogen, —OR6, N(R6)(R7), or a radical of the formula 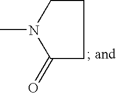
R1 is a radical of the formula 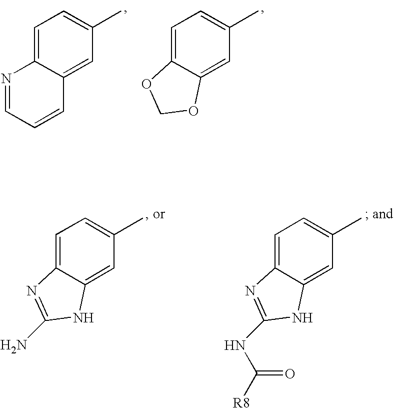
R8 is a radical of the formula 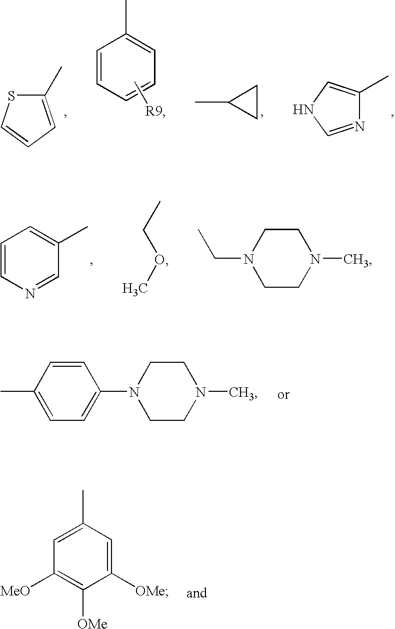
R9 is —NH(C═O)CH₃, —SO₂NH₂, —SO₂N(R6)(R7); and
R6 and R7 are independently C₁₋₆alkyl.

2. A compound of claim 1 in which R1 is a radical of the formula

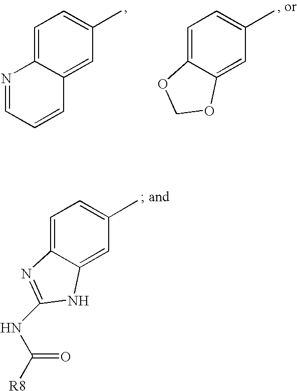

3. A compound of claim 2 in which R1 is a radical of the formula

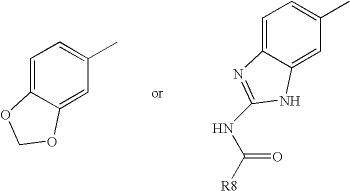

4. A compound of claim 3 in which R8 is 2-thienyl.

5. A method of inhibiting hYAK3 in a mammal; comprising, administering to the mammal a therapeutically effective amount of a compound of formula I, or a salt, solvate, or a physiologically functional derivative thereof 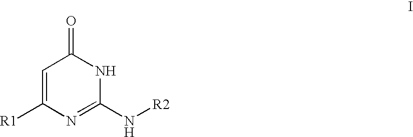
in which
R2 is a radical of the formula 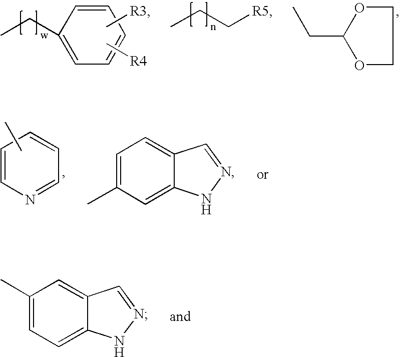
in which
n=1-2;
w=0-2;
R3 and R4 are independently hydrogen, hydroxy, —OR6, halogen, —SO₂NH₂, —OH, —C₁₋₆alkyl, —(C═O)—OEt, —NH(C═O)—CH₃, or a radical of the formula 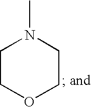
R5 is —NH₂, hydrogen, —OR6, —N(R6)(R7), or a radical of the formula 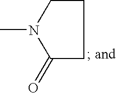
R1 is a radical of the formula 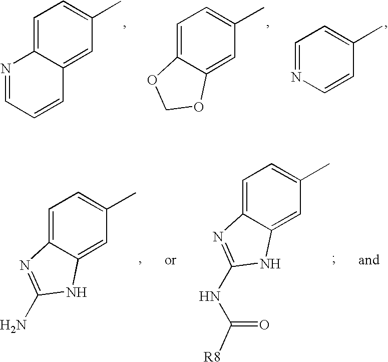
R8 is a radical of the formula 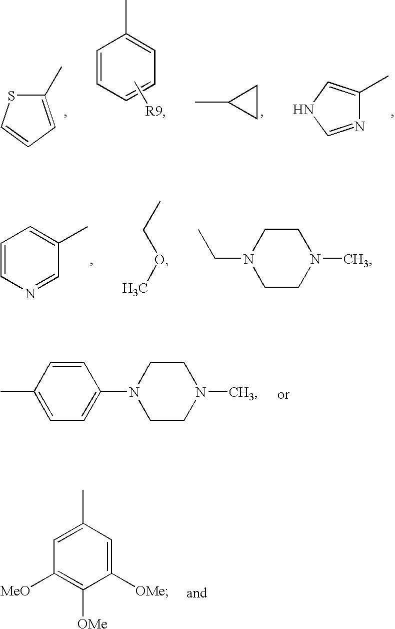
R9 is —NH(C═O)CH₃, —SO₂NH₂, —SO₂N(R6)(R7); and
R6 and R7 are independently C₁₋₆alkyl.

6. A method of treating or preventing diseases of the erythroid and hematopoietic systems, caused by the hYAK3 imbalance or inappropriate activity; comprising, administering to a mammal a therapeutically effective amount of a compound formula I, or a salt, solvate, or a physiologically functional derivative thereof 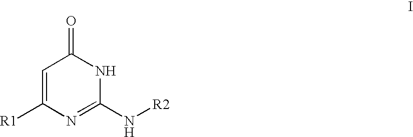
in which
R2 is a radical of the formula 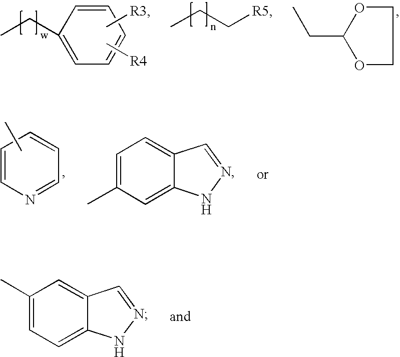
in which
n=1-2;
w=0-2;
R3 and R4 are independently hydrogen, hydroxy, —OR6, halogen, —SO₂NH₂, —OH, —C₁₋₆alkyl, —(C═O)—OEt, —NH(C═O)—CH₃, or a radical of the formula 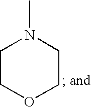
R5 is —NH₂, hydrogen, —OR6, —N(R6)(R7), or a radical of the formula 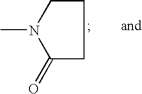
R1 is a radical of the formula 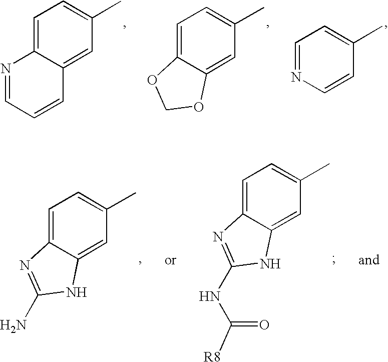
R8 is a radical of the formula 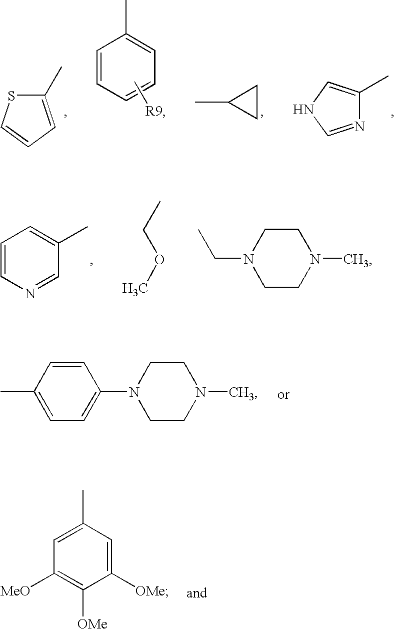
R9 is —NH(C═O)CH₃, —SO₂NH₂, —SO₂N(R6)(R7); and
R6 and R7 are independently C₁₋₆alkyl.

7. A method of claim 6 in which diseases of the erythroid and hematopoietic systems are selected from the group consisting of: anemia, aplastic anemia, myelodysplastic syndrome, myelosuppression, and cytopenia.

8. A method of treating or preventing diseases selected from the group consisting of: anemia, aplastic anemia, myelodysplastic syndrome, myelosuppression, and cytopenia; comprising, administering to a mammal a therapeutically effective amount of a compound formula I, or a salt, solvate, or a physiologically functional derivative thereof 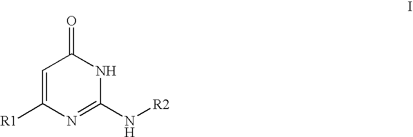
in which
R2 is a radical of the formula 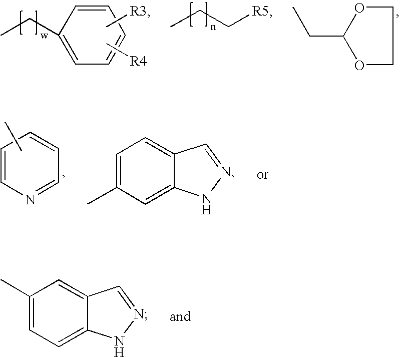
in which
n=1-2;
w=0-2;
R3 and R4 are independently hydrogen, hydroxy, —OR6, halogen, —SO₂NH₂, —OH, —C₁₋₆alkyl, —(C═O)—OEt, —NH(C═O)—CH₃, or a radical of the formula 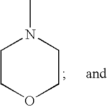
R5 is —NH₂, hydrogen, —OR6, —N(R6)(R7), or a radical of the formula 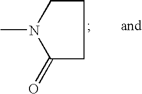
R1 is a radical of the formula 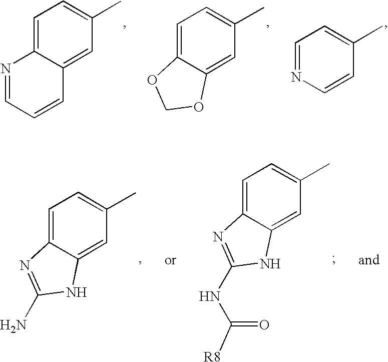
R8 is a radical of the formula 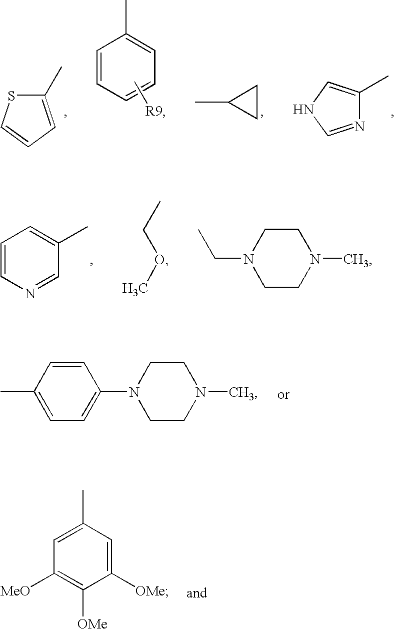
R9 is —NH(C═O)CH₃, —SO₂NH₂, —SO₂N(R6)(R7); and
R6 and R7 are independently C₁₋₆alkyl.

9. A pharmaceutical composition including a therapeutically effective amount of a compound formula I, or a salt, solvate, or a physiologically functional derivative thereof, and one or more pharmaceutically acceptable carriers, diluents and excipients 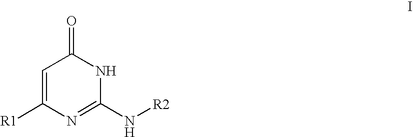
in which
R2 is a radical of the formula 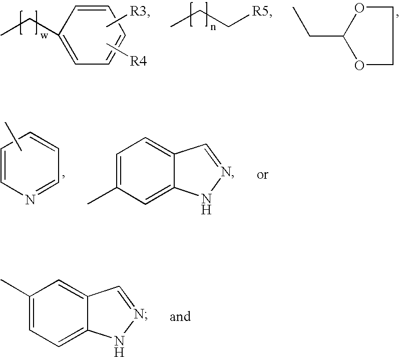
in which
n=1-2;
w=0-2;
R3 and R4 are independently hydrogen, hydroxy, —OR6, halogen, —SO₂NH₂, —OH, —C₁₋₆alkyl, —(C═O)—OEt, —NH(C═O)—CH₃, or a radical of the formula 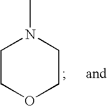
R5 is —NH₂, hydrogen, —OR6, —N(R6)(R7), or a radical of the formula 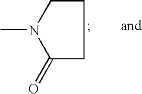
R1 is a radical of the formula 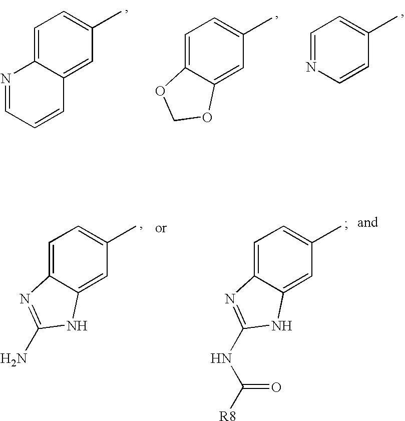
R8 is a radical of the formula 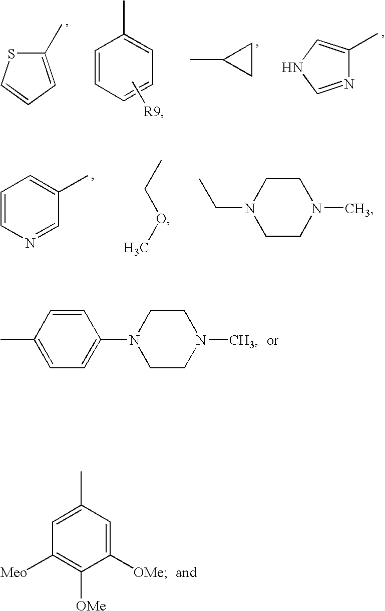
R9 is —NH(C═O)CH₃, —SO₂NH₂, —SO₂N(R6)(R7); and
R6 and R7 are independently C₁₋₆alkyl.

10. A compound selected from the group consisting of:
2-(2-Amino-ethylamino)-6-quinolin-6-yl-3H-pyrimidin-4-one;
2-(2-Dimethylamino-ethylamino)-6-quinolin-6-yl-1H-pyrimidin-4-one;
2-(3-Methoxy-benzylamino)-6-quinolin-6-yl-1H-pyrimidin-4-one;
2-{[2,6-Bis(methyloxy)phenyl]amino}-6-(6-quinolinyl)-4(1H)-pyrimidinone;
N-(5-{2-[(2-Chlorophenyl)amino]-6-oxo-1,6-dihydro-4-pyrimidinyl}-1H-benzimidazol-2-yl)-2-thiophenecarboxamide;
N-{5-[6-Oxo-2-(4-pyridinylamino)-1,6-dihydro-4-pyrimidinyl]-1H-benzimidazol-2-yl}-2-thiophenecarboxamide;
N-[5-(2-{[3-(Aminosulfonyl)phenyl]amino}-6-oxo-1,6-dihydro-4-pyrimidinyl)-1H-benzimidazol-2-yl]-2-thiophenecarboxamide;
N-{5-[6-Oxo-2-(phenylamino)-1,6-dihydro-4-pyrimidinyl]-1H-benzimidazol-2-yl}-2-thiop henecarboxamide;
N-(5-{2-[(3-Hydroxyphenyl)amino]-6-oxo-1,6-dihydro-4-pyrimidinyl}-1H-benzimidazol-2-yl)-2-thiophenecarboxamide;
N-{5-[2-(1H-Indazol-6-ylamino)-6-oxo-1,6-dihydro-4-pyrimidinyl]-1H-benzimidazol-2-yl}-2-thiophenecarboxamide;
N-[5-(2-{[4-(Methyloxy)phenyl]amino}-6-oxo-1,6-dihydro-4-pyrimidinyl)-1H-benzimidazol-2-yl]-2-thiophenecarboxamide;
N-[5-(2-{[2-(Methyloxy)ethyl]amino}-6-oxo-1,6-dihydro-4-pyrimidinyl)-1H-benzimidazol-2-yl]-2-thiophenecarboxamide;
N-[5-(2-{[2-(Methyloxy)phenyl]amino}-6-oxo-1,6-dihydro-4-pyrimidinyl)-1H-benzimidazol-2-yl]-2-thiophenecarboxamide;
N-[5-(2-{[3-(Methyloxy)phenyl]amino}-6-oxo-1,6-dihydro-4-pyrimidinyl)-1H-benzimidazol-2-yl]-2-thiophenecarboxamide;
N-[5-(2-{[3-(Dimethylamino)propyl]amino}-6-oxo-1,6-dihydro-4-pyrimidinyl)-1H-benzimidazol-2-yl]-2-thiophenecarboxamide;
N-[5-(2-{[2-(2,4-Dichlorophenyl)ethyl]amino}-6-oxo-1,6-dihydro-4-pyrimidinyl)-1H-benzimidazol-2-yl]-2-thiophenecarboxamide;
N-[5-(2-{[2-(4-Morpholinyl)phenyl]amino}-6-oxo-1,6-dihydro-4-pyrimidinyl)-1H-benzimidazol-2-yl]-2-thiophenecarboxamide;
N-(5-{2-[(2-Fluorophenyl)amino]-6-oxo-1,6-dihydro-4-pyrimidinyl}-1H-benzimidazol-2-yl)-2-thiophenecarboxamide;
N-(5-{2-[(2,5-Dichlorophenyl)amino]-6-oxo-1,6-dihydro-4-pyrimidinyl}-1H-benzimidazol-2-yl)-2-thiophenecarboxamide;
N-{5-[2-(Ethylamino)-6-oxo-1,6-dihydro-4-pyrimidinyl]-1H-benzimidazol-2-yl}-2-thiophenecarboxamide;
N-(5-{2-[(2-Methylphenyl)amino]-6-oxo-1,6-dihydro-4-pyrimidinyl}-1H-benzimidazol-2-yl)-2-thiophenecarboxamide;
N-[5-(6-Oxo-2-{[3-(2-oxo-1-pyrrolidinyl)propyl]amino}-1,6-dihydro-4-pyrimidinyl)-1H-benzimidazol-2-yl]-2-thiophenecarboxamide;
N-(5-{2-[(1,3-Dioxolan-2-ylmethyl)amino]-6-oxo-1,6-dihydro-4-pyrimidinyl}-1H-benzimidazol-2-yl)-2-thiophenecarboxamide;
3-(Acetylamino)-N-(5-{2-[(2-chlorophenyl)amino]-6-oxo-1,6-dihydro-4-pyrimidinyl}-1H-benzimidazol-2-yl)benzamide;
N-(5-{2-[(2-Chlorophenyl)amino]-6-oxo-1,6-dihydro-4-pyrimidinyl}-1H-benzimidazol-2-yl)cyclopropanecarboxamide;
4-(Aminosulfonyl)-N-(5-{2-[(2-chlorophenyl)amino]-6-oxo-1,6-dihydro-4-pyrimidinyl}-1H-benzimidazol-2-yl)benzamide;
N-(5-{2-[(2-Chlorophenyl)amino]-6-oxo-1,6-dihydro-4-pyrimidinyl}-1H-benzimidazol-2-yl)-4-[(dipropylamino)sulfonyl]benzamide;
N-(5-{2-[(2-Chlorophenyl)amino]-6-oxo-1,6-dihydro-4-pyrimidinyl}-1H-benzimidazol-2-yl)-1H-imidazole-4-carboxamide;
N-(5-{2-[(2-Chlorophenyl)amino]-6-oxo-1,6-dihydro-4-pyrimidinyl}-1H-benzimidazol-2-yl)-3,4,5-tris(methyloxy)benzamide;
N-(5-{2-[(2-Chlorophenyl)amino]-6-oxo-1,6-dihydro-4-pyrimidinyl}-1H-benzimidazol-2-yl)-3-pyridinecarboxamide;
N-(5-{2-[(2-chlorophenyl)amino]-6-oxo-1,6-dihydro-4-pyrimidinyl}-1H-benzimidazol-2-yl)-2-(methyloxy)acetamide;
N-(5-{2-[(2-Chlorophenyl)amino]-6-oxo-1,6-dihydro-4-pyrimidinyl}-1H-benzimidazol-2-yl)-2-(4-methyl-1-piperazinyl)acetamide;
4-(4-Methyl-1-piperazinyl)-N-{5-[6-oxo-2-(phenylamino)-1,6-dihydro-4-pyrimidinyl]-1H-benzimidazol-2-yl}benzamide;
6-(2-Amino-1H-benzimidazol-5-yl)-2-({[3-(methyloxy)phenyl]methyl}amino)-4(1H)-pyrimidinone;
N-{5-[2-({[3-(Methyloxy)phenyl]methyl}amino)-6-oxo-1,6-dihydro-4-pyrimidinyl]-1H-benzimidazol-2-yl}-2-thiophenecarboxamide;
6-Benzo[1,3]dioxol-5-yl-2-(3-methoxy-benzylamino)-1H-pyrimidin-4-one;
6-(1,3-Benzodioxol-5-yl)-2-(1H-indazol-5-ylamino)-4(1H)-pyrimidinone;
6-(1,3-Benzodioxol-5-yl)-2-{[3-(methyloxy)phenyl]amino}-4(1H)-pyrimidinone;
3-{[4-(1,3-Benzodioxol-5-yl)-6-oxo-1,6-dihydro-2-pyrimidinyl]amino}benzenesulfonamide;
6-(1,3-Benzodioxol-5-yl)-2-{[2-(methyloxy)phenyl]amino}-4(1H)-pyrimidinone;
2-[(2-Chlorophenyl)amino]-6-(4-pyridinyl)-4(1H)-pyrimidinone;
2-(1H-Indazol-6-ylamino)-6-(4-pyridinyl)-4(1H)-pyrimidinone;
3-{[4-Oxo-6-(4-pyridinyl)-1,4-dihydro-2-pyrimidinyl]amino}benzenesulfonamide;
2-{[3-(methyloxy)phenyl]amino}-6-(4-pyridinyl)-4(1H)-pyrimidinone;
2-(Phenylamino)-6-(4-pyridinyl)-4(1H)-pyrimidinone;
Ethyl 3-{[4-oxo-6-(4-pyridinyl)-1,4-dihydro-2-pyrimidinyl]amino}benzoate;
4-{[4-Oxo-6-(4-pyridinyl)-1,4-dihydro-2-pyrimidinyl]amino}benzenesulfonamide;
6-(4-pyridinyl)-2-(4-pyridinylamino)-4(1H)-pyrimidinone; and
N-(3-{[4-Oxo-6-(4-pyridinyl)-1,4-dihydro-2-pyrimidinyl]amino}phenyl)acetamide.
